(19) United States
(12) Patent Application Publication
(10) Pub. No.: US 2012/0079614 A1
(43) Pub. Date: Mar. 29, 2012

(54) CD109 POLYPEPTIDES AND USES THEREOF FOR THE TREATMENT OF SKIN CELLS

(75) Inventors: Anie Philip, Montreal (CA); Joshua Vorstenbosch, Montreal (CA); Carter Li, Pierrefonds (CA); Kenneth Finnson, Montreal (CA); Hahn Soe-Lin, Rockville, MD (US); Xiao-Yong Man, Montreal (CA); Albane Bizet, Montreal (CA); Hasan Al-Ajmi, Montreal (CA)

(73) Assignee: The Royal Institution for the Advancement of Learning/McGill University, Montreal (CA)

(21) Appl. No.: 13/148,273

(22) PCT Filed: Feb. 19, 2010
(86) PCT No.: PCT/CA10/00183

§ 371 Date: Dec. 19, 2011

Related U.S. Application Data
(60) Provisional application No. 61/150,953, filed on Feb. 9, 2009.

(51) Int. Cl. A61K 38/17; C07K 14/705; A01K 67/027; A61P 17/06; C12Q 1/68; A61P 17/00; A61P 17/02; A61K 8/64; G01N 33/567

(52) U.S. Cl. 800/18; 514/18.6; 514/18.7; 514/17.2; 514/18.8; 530/350; 800/14; 435/7.21; 435/6.12

(57) ABSTRACT

The invention concerns compounds, compositions and methods for the treatment of skin cells. Described herein are CD109 polypeptides and uses thereof for the in vivo treatment of various skin disorders, including skin fibrosis, skin scarring, wound healing and psoriasis.

### FIELD OF THE INVENTION

The invention relates to the field of dermatology. It concerns compounds, compositions and methods for using same in the treatment of skin cells, more particularly for reducing skin fibrosis, reducing skin scarring and/or promoting wound healing and treating psoriasis.

### BACKGROUND OF THE INVENTION

Transforming Growth Factor-β (TGF-β) is a 25 kDa multifunctional growth factor which plays a central role in the wound healing process and has clinical implications in many skin disorders including hypertrophic scarring, keloids, psoriasis and scleroderma. Aberrant TGF-β expression and signaling has been documented in each of the aforementioned pathologies. It is an important regulator of the immune response, angiogenesis, re-epithelialization, extracellular matrix (ECM) protein synthesis and remodeling.

Tam et al. discovered in 1998 a novel TGF-β1 binding protein, which they designated as r150 on keratinocyte skin cells (Tam, B. et al., Journal of Cellular Biochemistry, 1998. 70: 573-586). r150 was shown to bind TGF-β1, form a heteromeric complex with the TGF-β signaling receptors and inhibit downstream TGF-β induced responses (Tam, B. et al., Journal of Biological Chemistry, 2003. 278(49): 49610-49617; Finnson et al., FASEB J, 2006. 20(9): 1525-7; U.S. Pat. No. 7,173,002). Molecular cloning and subsequent sequencing was performed and r150 was identified as CD109 (Finnson et al., FASEB J, 2006. 20(9): 1525-7), a member of the α2-macroglobulin (α2M)/complement superfamily expressed on endothelial cells, platelets, activated T-cells, and a variety of tumors (Solomon, K. R., et al., Gene, 2004. 327(2): 171-83).

The nucleic acid and polypeptide sequences of CD109 are described in U.S patent publication US 2004/0266990, which teaches that CD109 is useful as a protease inhibitor and that CD109 may be administered to humans for treating various types of blood-related diseases or disorders. Although CD109 is getting more and more attention, the function of this protein remains largely unknown. For instance, prior to the present invention, no transgenic animal overexpressing CD109 had ever been produced and there had been no indication of a potential utility of CD109 for the treatment of skin cells or skin disorders, let alone its possible use in the treatment of scarring, keloid scarring, wound healing, hypertrophic scarring, fibrosis, psoriasis and scleroderma.

In normal skin, the epidermis and the dermis form a protective barrier against the external environment. Wound healing, or wound repair, is an intricate process in which the skin repairs itself after an injury. Once the protective barrier is broken, the normal physiological process of wound healing is immediately set in motion. Severely damaged skin often leads to excessive/abnormal scarring. Scars can and do influence patients' quality of life. Patients with scars may experience a disruption of daily activities, sleeping problems, anxiety and depression with consequent difficulties of social acceptance. Each year 100 million patients acquire scars, a proportion of which will become abnormal. There is thus a need for effective methods and composition promoting wound healing.

Hypertrophic scarring is a pervasive medical problem which often occurs as a result of burn, trauma, or surgical injury. Hypertrophic scarring is characterized by the formation of rigid scar tissue often resulting in significant functional impairment, leading to joint contractures, deformities, and central nervous system dysfunction. Keloid and hypertrophic scars are reported as a significant burden for the patients who face physical, psychological, esthetic and social consequences associated with significant financial costs. Keloid scars are thick, raised, itchy clusters of scar tissue that grow beyond the edges of a wound; they are more frequent in dark-skinned individuals and tend to recur. Hypertrophic scars are raised, red, thick scars that remains within the boundary of the injury. Keloid and hypertrophic scars result from local skin trauma or inflammatory skin disorders like lacerations, burns, skin piercing, surgery, etc. Treatment approaches include steroid injections into the scar, revision surgery, laser surgery, pressure garments and silicone dressings; efficacy is widely debated but generally limited. In spite of the stigma associated with scars, scar therapy is usually considered cosmetic. Unfortunately, there is no data on the incidence or prevalence of keloid and hypertrophic scars, other than that they are fairly common: in the USA it is estimated about 169 million scars are characterized as hypertrophic or keloid and 250,000 surgeries every year are related to scar revision. The most common complication for burn survivors is the development of abnormal scarring with an estimated rate of occurrence as high as 70%; we have thus used the incidence of burns to gain an estimate of the target population. Again, the data is scarce on the incidence of burns; the American Burn Association publishes data for the American market, but there are no pan-European databases providing data on burns and the same applies for the rest of the world. Estimates for the number of burns injury worldwide have been developed based on the reported number of annual cases of burns and scald of 1 in 754 in the USA and extrapolated to other countries; in addition the number of burns patients hospitalized was estimated from the rate of hospital admissions for burns of 9/100 000 population.

These estimates are probably underestimated as the American Burn Association reported 500,000 Americans and globally 6 million people suffered burns in 2007. In third world countries the incidence of burns is higher and under reported. With respect to hospitalized burns patients, the numbers are also likely underreported for the industrialized countries, but overrated for the third world countries as burns are treated in the home setting due to the lack of hospitals and dedicated burn units. The scarring process that results in abnormal scars is not well understood, cannot be prevented and is not successfully treated to meet the patient's expectations. Currently, no scar can be completely removed and new pharmaceutical compounds are highly needed because there is no effective treatment for hypertrophic scarring. Revision surgeries, pressure garments worn 23 hours a day for months, corticosteroid injections or silicone dressings have limited efficacy and today patients are coached not to have unrealistic expectations. Scar contractures are frequent after burn injuries across joints or skin concavities. They restrict the patient's movements, are typically disabling and dysfunctional and need to be surgically corrected; scars should thus not be considered only as cosmetic issue. There are both medical and human needs to improve abnormal scars.

Scleroderma (Systemic sclerosis, SSc) is a connective tissue disorder characterized by excessive extracellular matrix (ECM) synthesis and deposition in the skin and internal organs, leading to organ dysfunction and failure. Other features of SSc include autoimmunity and inflammation, widespread vasculopathy (blood vessel damage) affecting multiple vascular beds and progressive interstitial and perivascular fibrosis. Depending on its location and extent, localized scleroderma may cause severe cosmetic problems as well as restricted joint motion secondary to contractures. It is estimated that approximately 250 people per million have some form of scleroderma. Scleroderma (systemic and localized) affected an estimated 300,000 Americans in 2006 with women being four times more likely to develop the disease than men. It is estimated that a similar number of Europeans suffer from scleroderma. Treatment of localized scleroderma is generally of cosmetic concern unless they are associated with functional and cosmetic deformities. Scleroderma is treated with UVA phototherapy, vitamin D analogs, interferon γ and α, methotrexate, penicillamine, corticosteroids, immune suppressants such as tacrolimus, cyclosporine and biologics such as etanercept, drugs that are typically used in the treatment of psoriasis and/or RA. Such a variety of treatments clearly highlights the lack of effective therapy. There is no cure for SSc and no therapies currently available that reverse or decrease the progressive fibrotic process in this disease. Any agent that diminishes the excessive deposition of ECM proteins in SSc is of potential therapeutic benefit.

Psoriasis is a debilitating and disfiguring disease that confers unfavorable cardiovascular prognosis and affects ˜1-3% of the population. A large body of evidence indicates that TGF-β signaling is aberrantly regulated in psoriasis suggesting that manipulating TGF-β action in this disease may provide therapeutic benefit.

Existing treatments are not curative and only temporarily alleviate disease symptoms. These medications are often expensive and have variable side effects ranging from mild to severe. Thus, effective therapies for the afflicted patients are critically needed.

There is thus a need for methods and compositions for the treatment of skin cells and more particularly for methods and compounds for the promotion of wound healing, and for the treatment of scarring, fibrosis, psoriasis and scleroderma.

There is also a need for biomarkers for the diagnosis and monitoring of skin disorders, and more particularly for psoriasis and/or scleroderma in human subjects.

There is also a need for research tools, such as recombinant proteins and transgenic animals that may provide the means for more vigorous investigation of molecular pathogenesis of these skin-related diseases and conditions.

The present invention addresses these needs and other needs as it will be apparent from review of the disclosure, drawings and description of the features of the invention hereinafter.

### BRIEF SUMMARY OF THE INVENTION

The present invention contemplates compounds, compositions, and methods in the treatment of skin cells, more particularly for reducing skin fibrosis, reducing skin scarring and/or promoting wound healing and treating psoriasis.

The invention encompasses uses of a therapeutically effective amount a CD109 polypeptide for the treatment of skin cells in a human subject in need thereof and uses of a CD109 polypeptide in the manufacture of a medicament for the treatment of skin cells of a human subject in need thereof.

One aspect of the invention concerns a method for the in vivo treatment of skin cells of a mammalian subject in need thereof, the method comprising administering to the subject a therapeutically effective amount of a CD109 polypeptide. The CD109 polypeptide may comprise an amino acid sequence selected from the group consisting of SEQ ID NO:2, SEQ ID NO:4, SEQ ID NO:6; and therapeutically active fragments thereof. In preferred embodiments, the subject is afflicted with a skin disorder and treatment of skin cells comprises at least one of reducing skin fibrosis, reducing skin scarring and promoting wound healing.

One particular aspect of the invention concerns a method for the in vivo treatment of scarring in a human subject in need thereof, the method comprising contacting skin cells of the subject with a therapeutically effective amount of CD109 polypeptide. Preferably, the in vivo treatment comprises decreasing fibronectin levels and/or inhibiting fibronectin expression in the skin cells of the human subject. The scarring may derive from a burn, a trauma, a surgical injury or a chronic disease.

Another particular aspect of the invention concerns a method for promoting in vivo healing of a wound of a mammalian subject in need thereof. The wound may derive from a burn, a trauma, a surgical injury or a chronic disease.

Another particular aspect of the invention concerns a method for the in vivo treatment of a chronic inflammatory skin disorder in a human subject in need thereof, the method comprising contacting skin cells of the subject with a therapeutically effective amount of CD109 polypeptide.

Another particular aspect of the invention concerns a method for the in vivo treatment of a skin disorder associated with thickening and/or hardening of the skin of a human subject in need thereof, the method comprising contacting skin cells of the subject with a therapeutically effective amount of CD109 polypeptide.

The invention also relates to a composition for application to the skin of a mammalian subject in need thereof, the composition comprising a therapeutically effective amount of a CD109 polypeptide for the treatment of skin cells, and a pharmaceutically acceptable vehicle.

The invention further relates to a cosmetic composition for application onto the skin of a human subject, the composition comprising a cosmetically acceptable vehicle and a CD109 polypeptide capable of reducing skin fibrosis, reducing skin scarring and/or promoting wound healing.

The invention also relates to a method for the diagnostic of a skin disorder in a human subject, comprising assessing expression of a CD109 polypeptide in a skin sample from the subject.

Additional aspects of the invention concerns isolated or purified polypeptides, isolated or purified nucleic acid molecules, cell lines and non-human transgenic mammals.

An advantage of the present invention is that it provides compounds and methods for addressing skin disorders and conditions, especially those with excessive fibrosis such as hypertrophic scarring, keloids and SSc and in abnormal wound healing and psoriasis where cellular proliferation and differentiation are impaired. It also provides compositions which may have numerous health and cosmetic beneficial effects.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE DRAWINGS

FIG. 1A is a schematic diagram showing plasmid construct used to create CD109 transgenic mice, as described in Examples 1 and 2. The CD109 gene was cloned downstream of the K14 promoter using Gateway™ Cloning Technology (Invitrogen, Carlsbad, Calif.). The K14 promoter spatially restricts expression to the basal keratinocytes and thus to the epidermis.

FIG. 1B shows the results of RT-PCR and Western blot analysis of CD109 mRNA and protein expression in skin tissue of transgenic mice and their wild-type littermates (Examples 1 and 2). As shown, CD109 is overexpressed in the skin of the transgenic mice (TG) as compared to the wild-type littermates (WT). For the RT-PCR, the primers are unique to the CD109 transgene and therefore do not recognize endogenous CD109.

FIGS. 2A and 2B are images showing Masson's trichrome staining of skin tissue from wild-type and CD109 transgenic mice at 21 days (FIG. 2A) or 28 days (FIG. 2B) post-injection of bleomycin or PBS, as described in Example 1. Mice were injected on alternating days for either 21 days or 28 days after the initial injection, and then sacrificed, and tissues prepared for histological analysis. Bleomycin-injected tissue from transgenic mice displays a more organized collagen deposition as compared to bleomycin-injected wild-type littermates (arrows).

FIG. 3 is a bar graph showing that bleomycin-injected skin of CD109 transgenic mice display reduced dermal thickening as compared to wild-type littermates (Example 1). CD109 transgenic mice demonstrate a significant decrease in dermal thickness at 28 days following bleomycin injections as compared to bleomycin-injected wild-type littermates.

FIG. 4 are panels showing that skin of bleomycin-injected CD109 transgenic mice display a reduced Smad2 phosphorylation, without altering total Smad2 levels, and a decrease in type I collagen production as compared to skin of bleomycin-injected wild-type littermates at Day 28 post-injection (Example 1). Total proteins were extracted from the harvested skin tissue and the levels of phosphoSmad2, Smad2 and type I collagen, CD109 and actin (loading control) were determined by Western blot analysis.

FIG. 5 is a bar graph showing excisional wound closure measurements in CD109 transgenic and wild-type littermates at day 7 and day 14 post-wounding (Example 2). There were no significant differences observed in the rate of wound closure between the WT and TG group at both 7 and 14 days post-wounding in the excisional wound model.

FIG. 6 are pictures of H&E staining of excisionally wounded mouse skin 7 or 14 days post-wounding (Example 2). Transgenic mice (TG) overexpressing CD109 in the skin show improved healing at day 7 and day 14 as compared to wild-type littermates. Note the reduced cellularity (stained nuclei) at the wound margins (indicated by arrows) as well as in the healed dermis consistent with a reduction in scarring. Furthermore, the dermal ECM organization is improved in the transgenic mice compared to wild-type controls.

FIG. 7A-D include bar graphs and pictures representing quantification of wound healing parameters (Example 2). FIG. 7A shows that wounds from CD109 transgenic mice display an increase in epidermal thickening during wound healing as compared to wild-type littermates suggesting that CD109 promotes keratinocyte proliferation. Transgenic (TG) and wild-type (WT) littermates were excisionally wounded and epidermal thickness was determined at 7 and 14 days post-wounding by histological examination. Quantitative analysis of epidermal thickness (pixels) is shown (left). The arrows indicate the epidermal thickness in TG and WT mice (right). FIG. 7B shows that wounds from CD109 transgenic mice display a decrease in epidermal gap size at day 7 post-wounding as compared to wild-type mice suggesting that CD109 decreases keratinocyte migration. Transgenic (TG) and wild-type (WT) littermates were excisionally wounded and epidermal gap was determined at 7 and 14 days (not shown) post-wounding by histological examination. Quantitative analysis of epidermal gap (pixels) is shown (left *P<0.05). The arrows indicate the epidermal gap in TG and WT mice (right). FIG. 7C shows that wounds from CD109 transgenic mice display a decrease in dermal thickness at 7 days post-wounding as compared to wild-type littermates suggesting that CD109 produced by epidermal keratinocytes inhibits growth of dermal fibroblasts. Transgenic (TG) and wild-type (WT) littermates were excisionally wounded and dermal thickness was determined at 7 and 14 days post-wounding by histological examination. Quantitative analysis of dermal thickness (pixels) is shown (left *P<0.05). The arrows indicate the dermal thickness in TG and WT mice (right). FIG. 7D shows that wound from CD109 transgenic mice display decrease in the amount of granulation tissue present during wound healing. Transgenic (TG) and wild-type (WT) littermates were excisionally wounded and granulation tissue formation was examined at 7 and 14 days (not shown) post-wounding by histological examination. Quantitative analysis of granulation tissue area (pixels) is shown (left *P<0.05). The line drawn surrounds the granulation tissue present in WT mice at day 14 post-wounding which is absent in CD109 transgenic mice (right).

FIGS. 8A and 8B include bar graphs representing neutrophil and macrophage content in excisional wounds (Example 2). FIG. 8A shows that wounds in CD109 transgenic mice display a reduction in the number of neutrophils present during wound healing: transgenic (TG) and wild-type (WT) littermates were excisionally wounded and the number of neutrophils present was examined at 7 and 14 days post-wounding by immunostaining for the neutrophil marker Ly6-Gr. Quantitative analysis of neutrophils per high power field (hpf) is shown (*P<0.05). FIG. 8B shows that wounds from CD109 transgenic mice show a decrease in the number of macrophages present during wound healing as compared to wounds from wild-type littermates. Transgenic (TG) and wild-type (WT) littermates were excisionally wounded and the number of macrophages present was examined at 7 and 14 days post-wounding by immunostaining for the macrophage marker F4/80. Quantitative analysis of macrophages per high power field (hpf) is shown (*P<0.05 at day 7 post-wounding).

FIGS. 9A and 9B are pictures showing that incisional wound from CD109 transgenic mice show reduced scarring at Day 7 (FIG. 9A) and Day 14 (FIG. 9B) as compared to wound from wild-type littermates (Example 2). Transgenic (TG) and wild-type (WT) littermates were incisionally wounded and scarring was examined at 7 (FIG. 9A) and 14 days (FIG. 9B) post-wounding by histological examination. Consistent with a reduction in scarring, at day 7 and day 14 the incisional wound of the TG mice show reduced dermal cellularity (as determined by density of stained nuclei) and a smoother epidermis as compared to wild-type littermates. The arrows indicate the original location of the incisional wound.

FIG. 10 is a schematic diagram showing the main structural features of the full-length CD109 protein and the corresponding amino acid positions. The sequence of Peptide A (amino acid 606-766) is indicated (Example 3).

FIG. 11 shows that CD109 is released from the cell surface by PIPLC treatment and that soluble CD109 (150 kDa protein) binds TGF-β1 (Example 3). Human keratinocytes (HaCaT cells) were treated without or with phosphatidylinositolphospholipase C (PIPLC) to release GPI-anchored proteins (CD109) into the culture media. The media was collected and incubated with [¹²⁵I]TGF-β1 in the presence or absence of unlabeled TGF-β1 to compete with [¹²⁵I]TGF-β1 for binding to soluble CD109. [¹²⁵I]TGF-β1 associated proteins were resolved by SDS-PAGE and visualized by autoradiography.

FIG. 12 shows that CD109 overexpression decreases TGF-β1-induced PAI-1 and fibronectin protein expression by human keratinocytes (Example 3). HaCaT cells were transfected with CD109 or empty vector (EV) and treated for 18 hours without or with 0, 5 or 50 pM of TGF-β1. Cell lysates were prepared and analyzed by Western blot to detect PAI-1, fibronectin and actin (loading control).

FIG. 13A shows that full-length CD109 protein and a CD109-based peptide (Peptide A) compete with [¹²⁵I]TGF-β1 for binding to keratinocyte cell surface TGF-β receptors (Example 3). Human keratinocytes (HaCaT cells) were affinity labeled with 100 pM [¹²⁵I]TGF-β1 in the absence or presence of a 100-, 250 or 500-fold excesses of unlabeled TGF-β1 (positive control), full-length recombinant CD109, Peptide A or GST (negative control). [¹²⁵I]TGF-β1-associated proteins were separated by SDS-PAGE and visualized by autoradiography. FIG. 13B shows that a CD109-based peptide (Peptide A corresponding to amino acid sequence 606-766 of CD109) competes with [¹²⁵I]TGF-β1-binding to its cell surface receptors (Example 3). Analysis was performed as described for FIG. 13A.

FIG. 14 shows that CD109 inhibits TGF-β-induced transcriptional activity in human keratinocytes. HaCaT cells were transfected with a TGF-β responsive luciferase reporter construct (CAGA₁₂-lux) and β-galactosidase to measure transfection efficiency. Transfected cells were treated for 18 hours without or with 0-50 pM TGF-β1. Cell lysates were prepared and analyzed for luciferase and β-galactosidase activities and data are presented as luciferase activity (luciferase/β-galactosidase).

FIGS. 15A-F are pictures showing CD109 expression in psoriatic versus normal skin sections (Example 4). Cryostat sections were incubated with IgG control (C, D), anti-CD109 (TEA2/16) antibody (E, F) or without primary antibody (A, B). Immunohistochemistry was performed on psoriatic (B, D, F) and adjacent normal (A, C, E) skin samples from 10 patients. No significant immunostaining is observed for both normal and psoriatic skin samples treated without primary antibody (A, B) or with IgG control (C, D). As compared to normal human skin (E), expression of CD109 was decreased in psoriatic skin (F). Interestingly, the intensity of immunostaining with anti-CD109 is predominant in the epidermis in psoriatic (E) and adjacent normal skin (F). The immunostaining intensity was determined by measuring the area of each skin section pictures at two distinct and fixed thresholds intensity and by calculating the average ratio of the area values obtained using ImagePro™, as described in the Methods.

FIG. 15G is a bar graph showing a quantification of the results of FIGS. 15A-F (Example 4). The results are represented quantitatively by the bars and illustrate the decrease of CD109 expression pattern in psoriatic skin compared to adjacent normal skin in 8 out of 10 patients (G). FIG. 15H shows the results of real-time RT-PCR of CD109 mRNA expression in psoriatic and normal skin. Despite the marked decrease in CD109 protein expression (G), psoriatic epidermis expresses CD109 mRNA at comparable levels to normal skin (H).

FIGS. 16A(i), 16A(ii), 16A(iii), 16B and 16C are panels showing that CD109 inhibits TGFβ signaling in human keratinocytes (N/TERT-1 and NE6E7 cell lines) (Example 4). FIG. 16A(i): The release of GPI-anchored proteins (PI-PLC treatment) or the addition of exogenous CD109 recombinant protein to the media results in decreased SMAD2 phosphorylation and increased STAT3 phosphorylation (pSTAT3), STAT3 expression and Bcl-2 expression in N/TERT-1 and NE6E7 keratinocytes. Such effect can be significantly reduced by the addition of exogenous TGFβ. FIG. 16A(ii) shows that PIPLC treatment leads to increased amount of CD109 in the media in a time-dependent manner FIG. 16A(iii) shows that STAT3 levels are not altered in the absence of PIPLC treatment during the course of the experiment (note: 16(ii) and 16(iii) are controls for 16(i)0. FIG. 16B: while the treatment of N/TERT-1 cells with the TGFβ peptide results in a modest upregulation/stabilization of the TGFβ type I receptor (TGF-β RI), treatment of these cells with the exogenous CD109 protein significantly reduces the levels of TGFβ RI. FIG. 16C: inhibition of the CD109 gene expression via siRNA downregulates the CD109 protein expression, increases Smad2 phosphorylation and decreases STAT3 and Bcl-2 protein expression.

FIG. 17A is a line graph and FIG. 17B is a bar graph illustrating assessment of CD109 function in N/TERT-1 human keratinocyte cells (Example 4). FIG. 17A: Addition of CD109 to the culture media modestly alleviates the TGFβ-mediated growth inhibition in the N/TERT-1 cells (solid line with squares: N/TERT-1 untreated; solid line with circle: N/TERT-1+TGF-β1; dashed line with circle: N/TERT-1+TGF-β1+CD109. FIG. 17B: N/TERT-1 cells treated with the CD109 protein have higher clonigenic potential than the control N/TERT-1 cells suggesting that CD109 promotes survival of human keratinocytes.

FIG. 18A shows that PIPLC treatment or addition of recombinant CD109 protein increases STAT3 protein expression in HaCaT cells. FIG. 18B shows that addition of recombinant CD109 protein has a proliferative effect on HaCaT cells (Example 4). FIG. 18A: The PI-PLC treatment or the addition of recombinant CD109 protein to the media results in the upregulation of STAT3 protein in HaCaT keratinocyte cells. PI-PLC treatment is not able to induce such STAT3 upregulation in HaCaT GPI Mutated cells, where CD109 protein is expressed only in low amounts on the cell surface. However, the addition of exogenous CD109 to the media reproduces the STAT3 upregulation in these mutated cells. FIG. 18B: Cell growth curves of parental and HaCaT GPI-anchor mutant cells (defective in GPI-anchor biosynthesis). Parental HaCaT cells grow at a higher rate than the HaCaT GPI mutant cells under standard conditions. The reduced growth of HaCaT GPI mutant cells can be moderately improved by supplementing the media with the recombinant CD109 protein.

FIG. 19 are pictures showing immunolocalization of CD109 in normal and SSc skin and cultured fibroblasts (Example 5). Both epidermis and dermis from normal (A) and SSc (B) skin, and cultured normal (C) and SSc (D) fibroblasts are stained with anti-CD109 antibody and a FITC-conjugated secondary antibody which is detected by immunofluorescence. The dermis and epidermis of SSc skin display increased CD109 levels (B) as compared to normal skin (A). CD109 signal (white arrows) localizes mainly in the plasma membrane (C, D) and partly in the cytoplasm. Scale bar: 50 μM.

FIGS. 20A, 20B and 20C are panels and a dot graph showing expression of CD109 levels in SSc fibroblasts compared with normal fibroblasts (Example 5). (A) RT-PCR shows that CD109 mRNA levels in SSc fibroblasts (n=7 patients) are similar to that of normal (n=7) fibroblasts. Agarose gel (1.5%) was stained with ethidium bromide. No band was detected in negative control sample. GAPDH serves as an internal control. (B) Representative Western blot shows that CD109 protein is upregulated in SSc dermal fibroblasts (n=6) as compared with normal dermal fibroblasts (n=4). (C) Semi-quantitative analysis of the densitometric values derived from two independent experiments. The Y-axis shows the ratio of the optical density of CD109 (band at 180 kDa) to that of its corresponding β-actin. P-values denote statistical differences of the means between normal and SSc fibroblasts by t-test (P<0.05).

FIG. 21 is a picture of a Western blot showing the effect of TGF-β1 on expression of CD109 and fibronectin (Example 5). Western blot results show that the expression of CD109 is not altered by TGF-β1 in both normal and SSc fibroblasts, but the production of fibronectin is increased by TGF-β1 at 5, 10, 25, 50 and 100 pM.

FIGS. 22A and 22B are pictures of Western blots showing the effects of CD109 siRNA on production of ECM (Example 5). Blocking of CD109 by transfection of CD109 siRNA into fibroblasts increases the production of fibronectin, collagen I and CTGF in normal and SSc fibroblasts as compared to control siRNA transfected cells.

FIG. 23A shows that CD109 siRNA increases phosphorylation of Smad2 and Smad3 in both SSc and normal fibroblasts as compared to control siRNA transfected cells. FIG. 23B shows the levels of phosphorylated Smad2 and Smad3 and total TGF-β type I receptor levels in SSc and normal fibroblasts prepared from normal (n=4) and SSc (n=6) fibroblasts (Example 5). (A) In normal and SSc fibroblasts, blocking of CD109 by transfection of CD109 siRNA into fibroblasts increases phosphorylation of Smad2 and Smad3, while not altering the protein levels of Smad2 and Smad3. β-actin serves as a loading control. (B) Smad2 phosphorylation is increased in fibroblasts from SSc patients fibroblasts as compared to fibroblasts from normal controls whereas Smad3 phosphorylation levels are similar in the two groups.

FIG. 24 is a panel showing the effect of recombinant CD109 protein on fibronectin, collagen I and CTGF production in normal and SSc fibroblasts (Example 5). Normal and SSc fibroblasts were serum-starved for 24 h and then incubated with recombinant CD109 protein at 1.0 nM with or without 100 pM of TGF-β1 for 24 h. The cell lysates were prepared and production of fibronectin, collagen I and CTGF was determined by Western blot. Addition of recombinant CD109 protein decreases fibronectin, collagen type I an CTGF protein expression in SSc and normal fibroblasts. Membranes were reprobed with anti-actin antibody to verify that equal amounts of protein were loaded in each lane.

FIG. 25 is a schematic model for the potential role of CD109 in SSc (Example 5). At early stage of SSc, a balance exits between fibrogenic factors and fibrostatic factors. However, this balance is disrupted with the progression of SSc. At the established and late stage of SSc, fibrogenic factors (such as TGF-β) are upregulated prominently. The fibrostatic factors (such as CD109) are also upregulated to adapt to the increase of fibrogenic factors, but this upregulation cannot overcome the effect of fibrogenic factors.

FIG. 26 shows that recombinant CD109 protein inhibits basal and TGF-β1-induced production of collagen type I in SSc skin fibroblasts (Example 6). SSc skin fibroblasts were stimulated for 24 hours without (−) or with (+) 100 pM TGF-β1 in the absence (−) or presence (+) of various concentrations (0-1.0 nM) of recombinant CD109 protein. Cell lysates were analyzed by Western blot to detect the indicated proteins.

FIG. 27 shows that recombinant CD109 protein inhibits basal and TGF-β1-induced production of extracellular matrix proteins in normal and SSc skin fibroblasts (Example 6). Normal and SSc skin fibroblasts were stimulated for 24 hours without (−) or with (+) 100 pM TGF-β1 in the absence (−) or presence (+) of 1 nM recombinant CD109 protein. Cell lysates were analyzed by Western blot to detect the indicated proteins.

FIG. 28 shows that recombinant CD109 protein inhibits TGF-β1-induced production of CTGF in normal and SSc skin fibroblasts (Example 6). Normal and SSc skin fibroblasts were stimulated for 24 hours without (−) or with (+) 100 pM TGF-β1 in the absence (−) or presence (+) of various concentrations (0-1.0 nM) recombinant CD109 protein. Cell lysates were analyzed by Western blot to detect the indicated proteins.

FIG. 29 shows that CD109 peptide (606-766, peptide A) (upper panel) but not GST peptide (lower panel) inhibits basal and TGF-β1-induced production of collagen type I in SSc skin fibroblasts (Example 6). SSc skin fibroblasts were stimulated for 24 hours without (−) or with (+) 100 pM TGF-β1 in the absence (−) or presence (+) of various concentrations (0-5.0 nM) of CD109 peptide (606-766, peptide A, upper panel) or GST control (lower panel). Cell lysates were analyzed by Western blot to detect the indicated proteins.

FIG. 30 shows that CD109 peptide (606-766, peptide A) (upper panel) but not GST control peptide (lower panel) inhibits TGF-β1-induced production of PAI-I in SSc and normal skin fibroblasts (Example 6). SSc and normal skin fibroblasts were stimulated for 24 hours without (−) or with (+) 100 pM TGF-β1 in the absence (−) or presence (+) of various concentrations (0-5.0 nM) of CD109 peptide (606-766, peptide A) or GST control. Cell lysates were analyzed by Western blot to detect the indicated proteins.

### DETAILED DESCRIPTION OF PREFERRED EMBODIMENTS

The present inventors have found that CD109 has important in vivo biological functions in skin cells and in skin tissues and that CD109 polypeptides are useful for the in vivo treatment of various skin disorders.

### CD109 Polypeptide

CD109 is a GPI-anchored TGF-β co-receptor which is known to exist as two isoforms: CD109, a protein of about 180 kDa (1445 amino acids, also referred to as CD109L) and CD109S, a protein of about 180 kDa (1428 amino acids). Both CD109S and CD109L can exist as 150 kDa forms due to proteolytic cleavage.

The human cDNA sequence of CD109 is represented as SEQ ID NO: 1 and is cited under NCBI Refseq #AY149920. The amino acid sequence of the CD109 protein is represented as SEQ ID NO: 2. The human cDNA sequence of CD109S is represented as SEQ ID NO: 3 and is cited under NCBI Refseq #AY788891. The amino acid sequence of the CD109S protein is represented as SEQ ID NO: 4. As used herein, the term “CD109 polypeptide” refers to an isolated or purified polypeptide which comprises an amino acid sequence of SEQ ID NO: 2, an amino acid sequence of SEQ ID NO: 4 or a therapeutically active fragment thereof. Therapeutically active fragments of CD109 include those polypeptides having at least one desirable biochemical property on skin cells, such as the biological properties listed hereinafter. Also included are polypeptides which comprise a putative TGF-β binding domain. In one particular embodiment, the therapeutically active fragment is a polypeptide consisting of the amino acid sequence of SEQ ID NO: 6. SEQ ID NO: 6 is a 161 amino acid polypeptide which corresponds to amino acids 606-766 of CD109 (SEQ ID NO: 2) and of CD109S (SEQ ID NO: 4). The cDNA sequence encoding for this 161 amino acid polypeptide is represented in SEQ ID NO: 5.

A CD109 polypeptide fragment according to the invention may comprise at least 10, 15, 25, 50, 75, 100, 250, 500, 750, 1000, 1250, 1400 or up to 1444 contiguous amino acids of any of SEQ ID NO:2. Preferred CD109 polypeptide fragments comprise a putative TGF-β binding domain and at least one desirable biochemical property on skin cells, such as the biological properties listed hereinafter.

According to additional embodiments, the invention encompasses the use of a CD109 polypeptide homolog, in replacement and/or in combination to a CD109 polypeptide as defined herein. A suitable CD109 homolog according to the invention may have 70%, 75%, 80%, 85%, 90%, 95%, 97%, 99% similarity or more, over the full length of SEQ ID NO:2, or SEQ ID NO:4. Preferred CD109 homologs comprise a putative TGF-β binding domain and at least one desirable biochemical property on skin cells, such as the biological properties listed hereinafter. CD109 polypeptides, fragments or homologs according to the invention may also be incorporated into fusion proteins comprising one or more additional functional domains (e.g. GFP, YFP, RFP, 6-HIS, GST, FLAG, HA, myc, etc.).

As used herein, the term “skin cells” refers to the cells which make and/or are found in the epidermis and/or the dermis of mammals. Typically, the epidermis (the outermost layer of the skin) is comprised mainly of keratinocytes, melanocytes, Langerhans cells and Merkels cells, whereas the dermis (the inner or deeper layer of the skin) consists mainly of connective tissue, fibroblasts, and blood vessels (endothelial cells) The invention encompasses the treatment of a single type and/or of a plurality of skin cells from the dermis and/or the epidermis.

According to various aspects of the invention, the CD109 polypeptide comprises at least one, preferably two or more, biochemical property(ies) which is(are) useful or desirable for the treatment of skin cells. Examples of biochemical properties according to the invention include, but are not limited to:
(i) promoting reduction of in vivo dermal thickness and/or decreasing in vivo dermal thickening in response to a bleomycin insult (e.g. see example 1);
(ii) enhancing collagen organization in a bleomycin-induced in vivo model of fibrosis (See example 1);
(iii) reducing and/or impeding onset of bleomycin-induced fibrosis in vivo (e.g. see example 1);
(iv) inhibiting in vivo production of collagen type I in the skin cells (e.g. see example 1);
(v) inhibiting production of collagen type I, fibronectin and/or CTGF production in in vitro cultured systemic sclerosis fibroblast (e.g. see example 5);
(vi) decreasing basal production and/or decreasing TGF-β1-induced production of collagen type I, fibronectin and/or CTGF in in vitro cultured normal fibroblasts and in in vitro cultured systemic sclerosis fibroblasts (e.g. see example 5);
(vii) decreasing excessive ECM in in vitro cultured systemic sclerosis fibroblasts (e.g. see example 5);
(viii) improving collagen organization and/or reducing wound cellularity in in vivo excisional wounds (e.g. see example 2);
(ix) promoting epidermal thickening and/or inhibiting fibroplasia in in vivo excisional wounds (e.g. see example 2);
(x) promoting keratinocyte proliferation and/or inhibiting keratinocyte migration in in vivo excisional wounds (e.g. see example 2);
(xi) reducing and/or inhibiting dermal thickening in in vivo excisional wounds (e.g. see example 2);
(xii) reducing granulation and/or promoting resolution of granulation in in vivo excisional wounds (e.g. see example 2);
(xiii) reducing in vivo recruitment of inflammatory cells to a wound (e.g. see example 2);
(xiv) decreasing number of macrophages and/or neutrophils in a wound in vivo (e.g. see example 2);
(xv) decreasing dermal cellularity and/or promoting granulation tissue resolution in in vivo incisional wounds (e.g. see example 2);
(xvi) promoting a smoother epidermis in in vivo incisional wounds (e.g. see example 2);
(xvii) binding to TGF-β1 ligand in vitro (e.g. see example 3);
(xviii) decreasing fibronectin levels and/or inhibiting fibronectin expression in in vitro cultured human keratinocytes (e.g. see example 3);
(xix) decreasing in in vitro cultured human keratinocytes TGF-β1 induced plaminogen-activator inhibitor-1 (PAI-1) expression (e.g. see example 3);
(xx) binding to TGF-β1, TGF-β2 and/or TGF-β3 in an in vitro surface plasmon resonance assay (e.g. see example 3);
(xxi) inhibiting TGF-β binding to type 1 and type 2 TGF-β receptors in an in vitro affinity assay (e.g. see example 3);
(xxii) binding to TGF-β1 in vitro (e.g. see example 3);
(xxiii) inhibiting TGF-β1-induced transcriptional activity in in vitro cultured human keratinocytes (e.g. see example 3);
(xxiv) downregulating TGF-β/Smad signaling in in vitro cultured human keratinocytes (e.g. see example 4);
(xxv) upregulating expression of signal transducer and activator of transcription 3 (STAT3) expression and/or upregulating expression of STAT3 phosphorylation in in vitro cultured human keratinocytes (e.g. see example 4); and
(xxvi) increasing proliferation and/or survival of in vitro cultured human keratinocytes (e.g. see example 4).

It is within the skill of those in the art to determine whether a CD109 polypeptide according to the invention possesses one or more of those biochemical properties. The exemplification section hereinafter provides numerous in vitro and in vivo methods and assays in which such properties have been evaluated.

Additional aspects of the invention concerns an isolated or purified polypeptide consisting of the amino acid sequence of SEQ ID NO:6, and isolated or purified nucleic acid molecule consisting of the nucleotide sequence of SEQ ID NO:5, and pharmaceutical or cosmetic compositions comprising the same.

### Methods of Uses

As indicated hereinbefore and exemplified hereinafter, a CD109 polypeptide according to the invention has beneficial therapeutic and pharmaceutical properties and therefore, may have useful pharmaceutical applications in the treatment of various skin diseases and conditions in mammalians subjects. Medical and pharmaceutical applications contemplated by the inventors include, but are not limited to, wound healing, scarring, hypertrophic scarring, keloid scarring, fibrotic disorder, psoriasis and scleroderma.

According to preferred embodiments, the CD109 polypeptide is used for treating skin cells of a mammalian subject in need thereof. The term “mammalian subject” includes mammals in which treatment of skin cells or skin tissue is desirable. The term “subject” includes domestic animals (e.g. cats, dogs, horses, pigs, cows, goats, sheeps), rodents (e.g. mice or rats), rabbits, squirrels, bears, primates (e.g., chimpanzees, monkeys, gorillas, and humans), and transgenic species thereof. Preferably, the mammalian subject is a human, more preferably a human patient in need of treatment for one or more areas of its skin (e.g. due to a disease or an injury). Examples of a skin disorders encompassed by the present invention include, but are not limited to, wound healing, scarring, hypertrophic scarring, keloid scarring, fibrotic disorder, psoriasis and scleroderma.

As used herein, the terms “treatment” or “treating” of a subject include the application or administration of a compound of the invention to a subject (or application or administration of a compound of the invention to a skin cell or skin tissue of a subject) with the purpose of stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

Accordingly, a related aspect of the invention concerns a method for the in vivo treatment of scarring in a human subject. In one embodiment, the method comprises administering to a human subject in need thereof a therapeutically effective amount of a CD109 polypeptide. In preferred embodiments, the therapeutically effective amount of the CD109 polypeptide is contacted with afflicted skin cells of the human subject to decrease fibronectin levels and/or to inhibit fibronectin expression in the skin cells. The method may be particularly useful for the treatment of scarring occurring from burns, trauma or surgical injuries, and also hypertrophic scarring, keloid scarring.

Another related aspect of the invention concerns a method for promoting in vivo healing of a wound in a human subject. In one embodiment, the method comprises administering to a human subject in need thereof a therapeutically effective amount of a CD109 polypeptide. In preferred embodiments, the therapeutically effective amount of the CD109 polypeptide is contacted with afflicted skin cells or the wound of the human subject to improve collagen organization and/or to reduce wound cellularity in the wound; to promote epidermal thickening and/or to inhibit fibroplasia in the wound; to promote keratinocyte proliferation and/or to inhibit keratinocyte migration in the wound; to reduce and/or inhibit dermal thickening in the wound; to reduce granulation and/or to promote resolution of granulation in the wound; to reduce and/or inhibit recruitment of inflammatory cells to the wound; to decrease macrophages and/or neutrophils presence in the wound; and/or to decrease dermal cellularity and/or to inhibit granulation in the wound of the human subject. As used herein the term “wound” encompasses acute wounds deriving from burns, trauma or surgical injuries and wounds deriving from chronic conditions including, but not limited to, diabetic foot ulcers, venous leg ulcers and pressure ulcers. Accordingly, the methods of the invention may be useful for the treatment of acute wounds and chronic wounds associated with diabetic foot ulcers, venous leg ulcers or pressure ulcers. The methods of the invention may also be useful to promote normal wound closure and prevent abnormal scarring such as hypertrophic or keloid scarring.

Another related aspect of the invention concerns a method for in vivo treatment of a chronic inflammatory skin disorder in a human subject. In one embodiment, the method comprises administering to a human subject in need thereof a therapeutically effective amount of a CD109 polypeptide. In preferred embodiments, the therapeutically effective amount of the CD109 polypeptide is contacted with afflicted skin cells of the human subject to increase proliferation and/or survival of keratinocytes in the skin of the human subject. CD109 polypeptides fragments may be particularly useful for the treatment of psoriasis.

Another related aspect of the invention concerns a method for the in vivo treatment of a skin disorder associated with thickening and/or hardening of the skin of a human subject. In one embodiment, the method comprises administering to a human subject in need thereof a therapeutically effective amount of a CD109 polypeptide. In preferred embodiments, the therapeutically effective amount of the CD109 polypeptide is contacted with afflicted skin cells of the human subject and promotes reduction of dermal thickness and/or decreases dermal thickening of the skin of the human subject; enhances collagen organization in the skin of the human subject; reduces and/or impedes onset of fibrosis in the skin of the human subject; and/or inhibits production of collagen type I in skin cells the human subject. The method may be particularly useful for the treatment of scleroderma, hypertrophic scarring and keloid scarring.

In certain conditions where excessive CD109 activity or its enhanced release from the cell surface is a problem (for example, as in psoriasis), neutralizing its activity or preventing its release from the cell surface or blocking its cellular synthesis will be a useful approach to treat the disease.

Therefore, certain aspects the invention concerns treatment methods comprising inhibiting or neutralizing CD109 activity, CD109 expression, and/or CD109 release in skin cells. For instance, neutralizing the activity of CD109 can be achieved by using neutralizing anti-CD109 antibodies, or by using CD109-specific siRNA or shRNA nucleic acid sequences or CD109-specific antisense nucleic sequences or CD109-specific antisense morpholino oligonucleotides or dominant negative mutants of CD109 or other CD109 antagonists.

The release of CD109 can be blocked by different means, including inhibitors of enzymes which are involved in the release or processing of CD109. This can be accomplished by inhibiting the activity of enzymes such as PIPLC, PIPLD, furin (furinase), or mesotrypsin or other proteases by decreasing their activity or levels by using siRNA, shRNA, antisense morpholino oligonucleotides or dominant negative mutants or other antagonists.

### Pharmaceutical and Cosmetic Compositions

Related aspects of the invention concerns cosmetic and pharmaceutical compositions comprising an effective amount a CD109 polypeptide of the invention described herein. One particular aspect concerns the use of a therapeutically effective amount of a CD109 polypeptide for the treatment of skin cells in a human subject in need thereof. Another particular aspect concerns the use of a CD109 polypeptide for the manufacture of a medicament for the treatment skin cells in a human subject in need thereof.

As used herein, the term “therapeutically effective amount” means the amount of compound that, when administered to a subject for treating or preventing a particular disorder, disease or condition, is sufficient to effect such treatment or prevention of that disorder, disease or condition. Dosages and therapeutically effective amounts may vary for example, depending upon a variety of factors including the activity of the specific agent employed, the age, body weight, general health, gender, and diet of the subject, the time of administration, the route of administration, the rate of excretion, and any drug combination, if applicable, the effect which the practitioner desires the compound to have upon the subject and the properties of the compounds (e.g. bioavailability, stability, potency, toxicity, etc.), and the particular disorder(s) the subject is suffering from. In addition, the therapeutically effective amount may depend on the subject's skin condition (e.g. lightly or severely burned, presence of other skin injuries, etc.), the severity of the disease state, or underlying disease or complications. Such appropriate doses may be determined using any available assays. When one or more of the compounds of the invention is to be administered to humans, a physician may for example, prescribe a relatively low dose at first, subsequently increasing the dose until an appropriate response is obtained.

“Pharmaceutically acceptable vehicle” or “cosmetically acceptable vehicle” refer to a diluent, adjuvant, excipient, or carrier with which a compound is administered. The terms “pharmaceutically acceptable” and “cosmetically acceptable” refer to drugs, medicaments, inert ingredients, etc., which are suitable for use in contact with the skin tissues of humans and lower animals without undue toxicity, incompatibility, instability, irritation, allergic response, and the like, commensurate with a reasonable benefit/risk ratio. It preferably refers to a compound or composition that is approved or approvable by a regulatory agency of the Federal or state government or listed in the U.S. Pharmacopoeia or other generally recognized pharmacopoeia for use in animals and more particularly in humans. The pharmaceutically or cosmetically acceptable vehicle can be a solvent or dispersion medium containing, for example, water, ethanol, polyol (for example, glycerol, propylene glycol, and liquid polyethylene glycol and the like), suitable mixtures thereof, and vegetable oils. Additional examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Prevention of the action of microorganisms in the composition can be achieved by various antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of injectable compositions can be brought about by including in the composition an agent which delays absorption, for example, aluminum monostearate or gelatin.

The compounds of the invention may be formulated prior to administration into pharmaceutical and/or cosmetic compositions using available techniques and procedures. In preferred embodiments, the compositions according to the invention are formulated for application to the skin (e.g. topical administration, subcutaneous (sc) injection or intra-dermal injection.

In preferred embodiments, the compound and compositions are administered topically to a subject, e.g. by using an impregnated wound dressing or by the direct laying on or spreading of the compound or composition on the epidermal or epithelial tissue of the subject, or transdermally via a “patch”. Such compositions include, for example, lotions, creams, solutions, gels and solids. These topical compositions may comprise an effective amount, usually at least about 0.1%, about 1%, about 5%, or 10% or more of a compound of the invention. Suitable carriers for topical administration typically remain in place on the skin as a continuous film, and resist being removed by perspiration or immersion in water. Generally, the carrier is organic in nature and capable of having dispersed or dissolved therein the therapeutic agent. The carrier may include pharmaceutically acceptable emollients, emulsifiers, thickening agents, solvents and the like.

The method of treatment of the present invention may also include co-administration of at least one compound according to the invention (e.g. a CD109 polypeptide), together with the administration of another therapeutically effective agent for the prevention and/or treatment of wounds, scarring, hypertrophic scarring, keloid scarring, fibrotic disorders, psoriasis and/or scleroderma.

In one embodiment, a compound of the invention (e.g. a CD109 polypeptide) is used in combination with at least one additional known compound which is currently being used or in development for treating skin cells and/or skin tissues. Examples of such known compounds include but are not limited to: vitamin D analogs, interferon γ and α, methotrexate, penicillamine, corticosteroids, immune suppressants such as tacrolimus, cyclosporine biologics such as etanercept, anti-infectives such as silver and iodine composition, ibuprofen and antibiotics.

### Biomarkers

The invention further relates to biomarkers, more particularly the use of CD109 as a biomarker for diagnosing psoriasis and scleroderma. As shown in Example 4 CD109 protein levels were markedly lower in the lesional skin of psoriasis patients as compared to normal skin. Similarly, as shown in Example 5, CD109 expression is markedly upregulated in scleroderma patient skin samples as compared to skin from normal subjects.

Accordingly, an additional aspect relates to a method for the diagnostic of a skin disorder in a human subject, comprising assessing expression of a CD109 polypeptide in a skin sample from the subject. In one embodiment, the CD109 polypeptide levels are lower in a lesional skin sample from a subject suffering from psoriasis as compared to normal skin (i.e. a skin sample from a healthy subject). In another embodiment the CD109 polypeptide levels are higher in a skin sample from a subject suffering from scleroderma as compared to a skin sample from a normal healthy subject.

As used herein the terms “assessing expression” is meant an assessment of the degree of expression of a marker in a sample at the nucleic acid or protein level, using technology available to the skilled artisan to detect a sufficient portion of any marker expression product (including nucleic acids and proteins). Any suitable method known in the art can be used to measure the marker's expression. For instance, assessment of the expression of the CD109 polypeptide according to the invention may comprise detecting and/or measuring le level of one or more marker expression products, such as mRNA and protein.

### Transgenic Animals

The invention encompasses vectors comprising a nucleic acid molecule encoding a CD109 polypeptide according to the invention. The invention also encompasses host cells transformed with such vectors and transgenic animals expressing, e.g. over expressing, a CD109 polypeptide of the invention. Those cells and animals could serve as models of disease in order to study the mechanism of the function of the CD109 gene and also allow for the screening of therapeutics.

Exemplary methods for producing host cells and transgenic animals according to the invention are provided herein in the exemplification section. Host cells include, but are not limited to, fibroblasts, keratinocytes, endothelial cells, HACAT cells and N/PERT-1 cells, NE6E7 cells, 293 cells, 293T cells, 293A cells, and CHO cells. Transgenic animals can be selected from farm animals (such as pigs, goats, sheep, cows, horses, rabbits, and the like), rodents (such as mice, rats, guinea pigs, mice, and the like), non-human primates (such as baboons, monkeys, chimpanzees, and the like), and domestic animals (such as dogs, cats, and the like).

### EXAMPLES

### Example 1

### Transgenic Mice Overexpressing CD109 in the Epidermis: Bleomycin-Induced Skin Fibrosis Mouse Model

Bleomycin-induced fibrotic mouse model has become a well recognized in vivo model for scleroderma and other fibrotic disorders (Yamamoto and Nishioka, Arch Dermatol Res, 2004. 295(10): p. 453-6). It has been well documented that TGF-β signaling has a strong impact on the onset of the fibrosis in this model (Lakos Am J Pathol, 2004. 165(1): p. 203-17). In the current study, transgenic mice overexpressing CD109 in the epidermis were generated and these mice and their control littermates were injected intradermally with bleomycin or vehicle control (PBS) to determine if CD109 affects fibrosis. By analyzing collagen organization, extracellular matrix deposition (ECM) and dermal thickness of both the wild-type and transgenic mice, we were able to show that CD109 significantly ameliorates the bleomycin-induced fibrotic response in vivo.

### Materials & Methods

Generation of Transgenic Mice: CD109 was cloned using Gateway™ technology into a vector containing the K14 promoter, which restricts transgene expression to basal keratinocytes. The transgene was then excised from the vector and injected into fertilized FVB mouse oocytes at the McGill Transgene Core Facility. Founder mice were identified by Southern Blot and PCR using primers unique to the transgene. Subsequent progeny were identified by PCR and CD109 expression was confirmed by both RT-PCR and Western blot.

Bleomycin Injections: Mice (12 transgenic and 12 wild-type) were anesthetized by inhalation of isofluorane and intradermally injected with 0.1 mL of bleomycin in PBS (1 mg/mL) or PBS alone (control) every other day at two different sites (cranial and caudal) on the dorsal skin surface of each animal as previously described (Yamamoto et al. J Invest Dermatol, 1999, 112(4):456-62). At both day 21 and day 28, 12 transgenic mice and 12 wild-type littermates (6 mice from each genotype injected with bleomycin and 6 from each genotype injected with PBS) were sacrificed and skin was harvested from both sites. Tissue from the cranial site was snap-frozen and used for Western blot analysis and tissue from the caudal site was fixed in formalin for histological analysis.

Histology Analysis: Mouse skin was fixed overnight in formalin and embedded in paraffin. 7 μm tissue sections were cut and mounted onto slides for H&E and Mason's Trichrome staining.

Determination of Dermal Thickness: Stained sections were photographed and measured using ImageProPlus6™ software from Media Cybernetics. Dermal thickness was determined as the distance between the basement membrane and the superficial aspect of the fatty deposits of the hypodermis. Five independent measurements of each section were taken and the mean values were determined to control for variability within each section.

Western Blot Analysis: Mouse skin was snap-frozen in liquid nitrogen and stored at −80° C. until it was homogenized in RIPA buffer. Protein concentration was determined using the Lowry method and equal amounts of protein were loaded on a 7.5% SDS-PAGE gel. Proteins were transferred to nitrocellulose membrane, blocked in 5% milk in TBST, and probed with antibodies (Collagen Type I, Abcam; β-actin, Santa Cruz; Fibronectin, Santa Cruz; phosphoSMAD 2/3, Santa Cruz; SMAD 2, Cell Signaling) that were detected using a secondary antibody (Cell Signaling) and treated with ECL™ reagent.

### Results

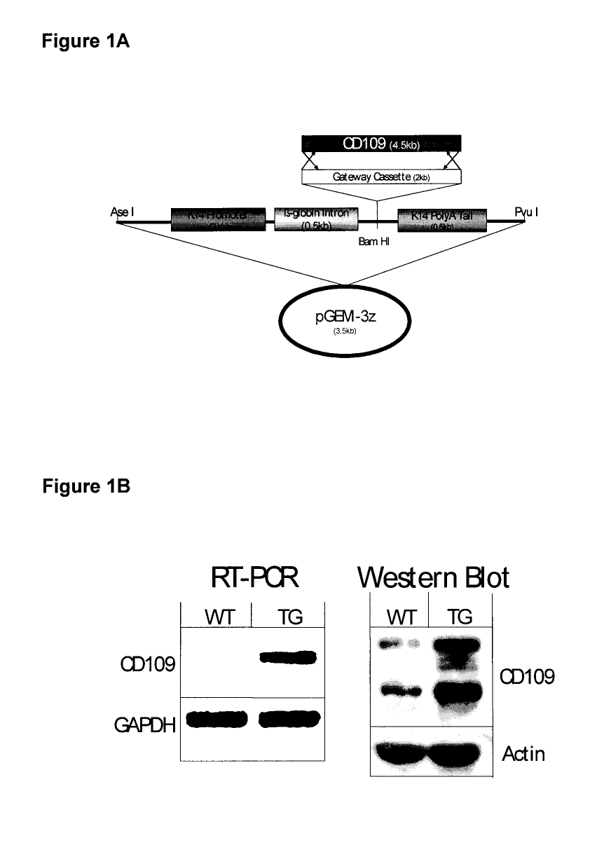

Generation of Transgenic Mice Overexpressing CD109 in the Epidermis: Transgenic mice overexpressing CD109 in the epidermis were generated for in vivo studies. As shown in FIG. 1, this was accomplished by cloning the human CD109 gene downstream of the keratin 14 promoter to restrict its expression to basal keratinocytes. Upon generation of these mice, incorporation of the transgene into the genome was confirmed, as determined by southern blot and PCR (data not shown). Additional verifications confirmed that the transgenic mice overexpress CD109 mRNA and protein in the skin.

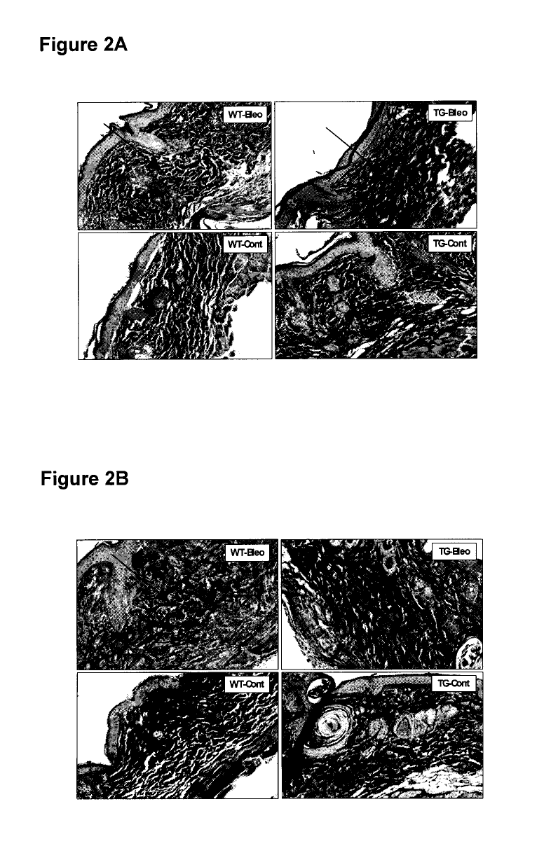

CD109 Decreases Bleomycin-Induced Skin Fibrosis: After confirming that CD109 was overexpressed in the skin, the effects of CD109 on bleomycin-induced skin fibrosis were determined. It has been shown that it takes approximately 28 days for bleomycin to induce complete fibrosis (Yamamoto et al., 1999. Journal of Investingative Dermatology. 112(4) 456-62). Therefore transgenic and wild-type mice were injected with bleomycin or PBS control for 21 and 28 days and the skin was analyzed at these time points. As shown in FIG. 2, bleomycin-injected CD109 transgenic mice display more densely packed and organized collagen fibrils as compared to bleomycin-injected wild-type littermates. Since disorganized collagen deposition is a hallmark of fibrosis, these data suggest that CD109 impedes the onset of fibrosis induced by bleomycin. This effect is more prominent after 28 days of bleomycin injection where transgenic mice display a ‘basket-weave’ pattern of collagen organization typical of normal skin whereas bleomycin-injected wild-type littermates exhibit nodular collagen deposition typical of severe fibrosis. No histological differences were observed by Masson's Trichrome staining of PBS-injected transgenic or wild-type mice indicating that the injections themselves had no effect on induction of fibrosis.

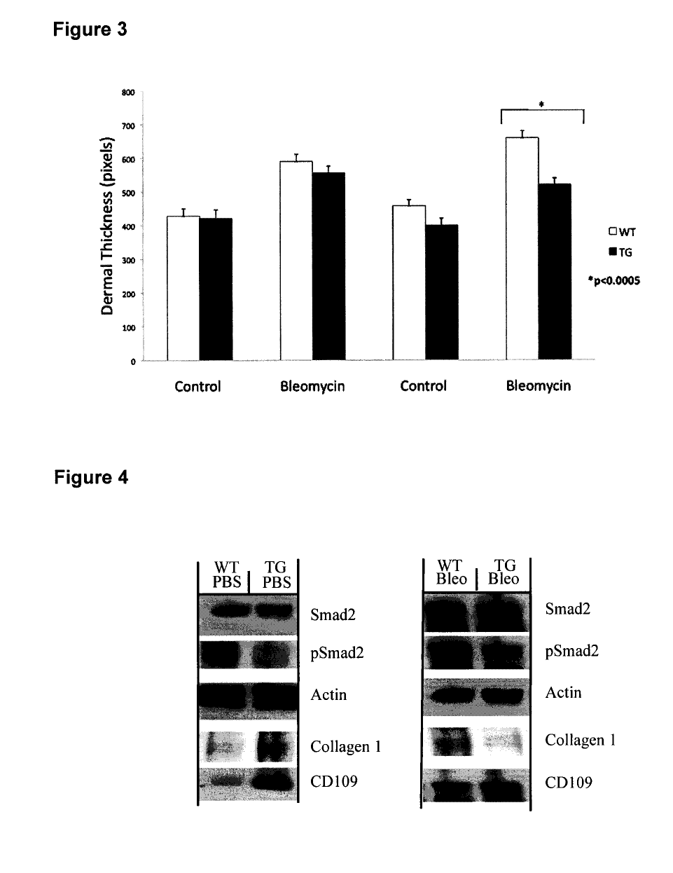

CD109 decreases bleomycin-induced dermal thickening: Many fibrotic disorders including scleroderma and hypertrophic scarring are characterized by a thickening of the skin. One of the causes for this thickening is proliferation of the dermis, in which the fibroblasts divide and deposit ECM and expand this layer of the skin. To determine whether CD109 can inhibit the thickening of the dermis, the dermal thickness (which we define as the distance between the basement membrane and the most superficial aspect of the fatty deposits of the hypodermis.) was assessed in bleomycin- and PBS-injected transgenic mice and their control littermates. As shown in FIG. 3, bleomycin-injected CD109 transgenic mice display reduced dermal thickness as compared to bleomycin-injected wild-type mice after 28 days of bleomycin injection, although no difference in dermal thickness was observed between transgenic and wild-type mice after 21 days of bleomycin injection. No notable differences were observed in dermal thickness in PBS-injected transgenic or wild-type mice confirming the histological results that the injections themselves do not contribute to the fibrotic process. These data suggest that CD109 decreases dermal thickening in vivo in response to bleomycin insult.

CD109 inhibits TGF-β Signaling and ECM Protein Expression: To determine whether the histological data were consistent with reductions in TGF-β signaling, the expression of ECM proteins and phosphorylation of Smad proteins was analyzed by western blot in tissues from bleomycin- and PBS-injected transgenic mice and wild-type littermates. As shown in FIG. 4, bleomycin- and PBS-injected CD109 transgenic mouse skin tissue display decreased Smad2 phosphorylation as compared to bleomycin- and PBS-injected wild-type littermate skin tissue, respectively. In addition, upon treatment with bleomycin, collagen I expression is decreased in the CD109 transgenic mice compared to the wild-type mice suggesting that CD109 inhibits fibrosis through a TGF-β dependent mechanism. Taken together, these data are consistent with the current histological results indicating diminished fibrosis in the transgenic mice, and suggest that CD109 may have therapeutic value for treatment of scleroderma and other fibrotic disorders.

### Discussion

This example demonstrates that CD109, when overexpressed in the skin, can ameliorate bleomycin-induced skin fibrosis in vivo. Fibrotic disorders such as scleroderma and hypertrophic scarring are characterized by excessive ECM deposition and irregular collagen organization (Aarabi et al., FASEB J, 2007. 21(12): 3250-61) similar to what can be observed here histologically in bleomycin-injected wild-type (control) mice injected with bleomycin. In the presence of overexpressed CD109 in the epidermis, more ‘basket-weave’ type of collagen organization is observed, which is consistent with normal organization in the skin. Collectively, these data suggest that CD109 can impede the progression of fibrosis, and thus have therapeutic potential in reversing the course of fibrotic diseases.

### Example 2

### Transgenic Mice Overexpressing CD109 in the Epidermis: Wound Healing Studies

Using established wound models, the current experiments were carried out to determine the effect of CD109 on various wound healing parameters and its abilities to reduce scarring. Theses results demonstrate that CD109 transgenic mice display more organized collagen and ECM deposition, reduced granulation tissue formation and decreased numbers of immune cells (macrophages and neutrophils) as compared to wild-type littermates suggesting that CD109 reduces inflammation, promotes granulation tissue resolution and improves scarring. Furthermore, CD109 did not affect the wound closure macroscopically, at the histological level, suggesting that it improves scarring without altering the normal wound healing response. Taken together, these results support CD109 therapeutic value in the treatment of pathological scarring such as keloid formation and hypertrophic scarring and in improving surgical scars.

### Materials & Methods

Generation of Transgenic Mice: Transgenic mice overexpressing CD109 were generated as described hereinbefore at Example 1.

Administration of Wounds:

Excisional wounds: Mice (6 wild-type and 6 transgenic at each time point) were anesthetized by inhalation of isofluorane and depilated by shaving and treatment with Nair™. Upon depilation, mice were excisionally wounded using a 5 mm biopsy punch, and sacrificed at 7 and 14 days post-wounding. The wounds were collected, with one wound being snap-frozen in liquid nitrogen for biochemical analysis and the other fixed overnight in formalin for histological analysis.

Incisional wounds: Mice were anesthetized as described above and 30 mm incisional wounds were created caudal to incisional wounds at the midline of the back of CD109 transgenic mice and wild-type littermates. Wounds were sutured closed using 5-0 silk sutures and harvested after 7 and 14 days. Tissue was fixed overnight in formalin for histological assessment.

Wound Closure Analysis: At wounding and sacrifice, a ruler was placed adjacent to the mouse to indicate size and excisional wounds were photographed using a digital camera. Wounds were analyzed using ImageJ™ software from NIH and wound area was calculated.

Histological Analysis: After fixing wound skin overnight in formalin, excisional and incisional wounds were processed and embedded in paraffin. Seven μm sections were then mounted on slides for subsequent histological analysis. For histological assessment, wounds were stained using hematoxylin and eosin and photographed using ImageProPlus6™ software from Media Cybernetics. Analysis of macrophage and neutrophil presence was performed in excisional wounds using immunohistochemistry with antibodies against F4/80 and Ly6-GR (eBioscience), respectively (Wang et al., Proc Natl Acad Sci USA, 1997. 94(1): p. 219-26). Briefly, tissue sections were deparaffinized in xylene and rehydrated through graded ethanol washes. Antigen retrieval was conducted by treatment with ProteinaseK/EDTA, and blocked for 1 hour using SuperBlock™ (Scytek) and incubated with antibodies overnight at 4° C. The following day, samples were treated with biotinylated secondary antibody (VectorLabs) for 1 hour at room temperature and then incubated for 1 hour with ABC™ detection solution (VectorLabs) and developed with ImpactDAB™ (VectorLabs), and counterstained with hematoxylin. Images were captured using ImageProPlus6™ software (Media Cybernetics) and immune cell number was determined by counting the number of cells positive for each marker per high power field.

Wound Parameter Analysis: Excisional wound sections stained with hematoxylin and eosin were photographed and measured using ImageProPlus6™ software from Media Cybernetics to determine epidermal thickness, epidermal gap, dermal thickness and granulation tissue area (Galiano et al., Wound Repair Regen, 2004. 12(4): p. 485-92). Five measurements from each section were performed and mean values were calculated to gain representative quantitative data comparing within and between groups. Epidermal thickness was measured as the distance from the top layer of keratinocytes to the bottom layer of keratinocytes at the wound edge. Epidermal gap was measured as the distance between the opposing epithelial edges approaching each other. Dermal thickness was measured as the distance from the basement membrane to the superficial aspect of the hypodermis at the wound edge. Granulation tissue area was measured as the area of the granular tissue observed within the wound bed.

### Results:

Generation of Transgenic Mice: As described previously in the Results section of Example 1, the transgenic mice overexpress CD109 mRNA and protein in the skin by transgene-specific RT-PCR and Western blot analysis, respectively (FIG. 1B).

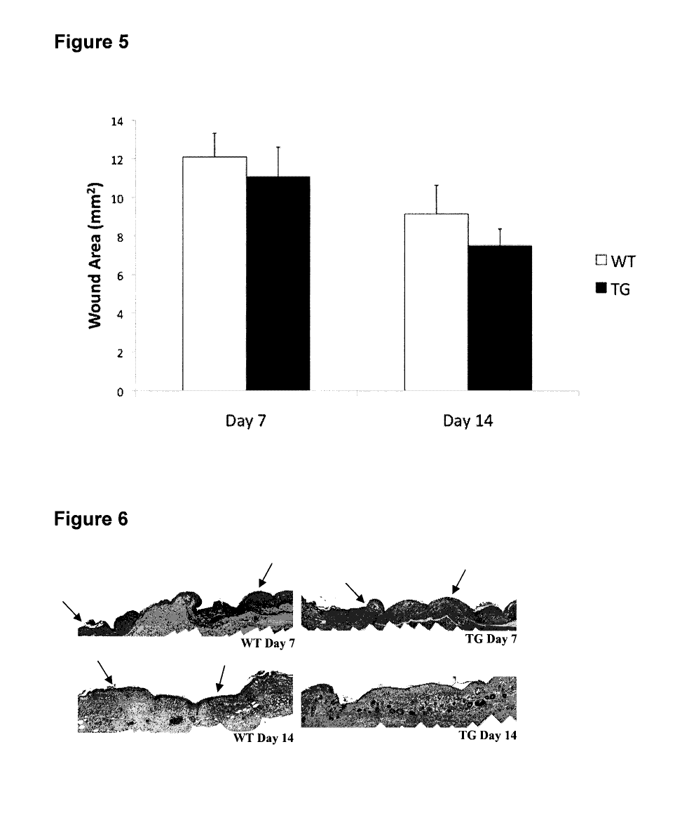

CD109 does not alter wound closure of excisional wounds: First, the experiment was carried out to determine whether CD109 overexpression in the epidermis affects the rate of wound closure using an excisional wound model. Excisional wounds were created on the dorsal surfaces of CD109 transgenic mice and wild-type littermates. The rate of wound closure was determined by photographing the wounds at 7 and 14 days post-wounding, digitally tracing the wound margins using ImageJ™ software and calculating wound areas. As shown in FIG. 5, wound closure in transgenic and wild-type mice were not significantly different. These data suggest that CD109 has no adverse effect on wound healing and is consistent with the notion that wound healing is associated with more endogenous TGF-β than what is needed for optimal wound healing and that decreasing excessive TGF-β activity at the wound site may reduce scarring.

CD109 improves collagen organization and reduces wound cellularity in excissional wounds: Next, the potential role of CD109 on wound healing parameters was examined by histological analysis. As shown in FIG. 6, excisional wounds in CD109 transgenic mice display a more organized collagen deposition as compared to wild-type littermate wounds. Furthermore, excisional wounds of CD109 transgenic mice display reduced cellularity in the wound site and in the healing dermis as compared to those in wild-type littermates. Because improved collagen deposition and decreased cellularity are hallmarks of a reduced scarring these data suggest that CD109 has anti-scarring potential.

CD109 promotes epidermal thickening and inhibits fibroplasia in excissional wounds: Wound healing is characterized by three overlapping phases: inflammation, proliferation, and maturation. To gain a better understanding of how CD109 affects these different phases, the epidermal thickness, epidermal gap, dermal thickness, and granulation tissue area was analyzed in excisional wounds of transgenic and wild-type mice.

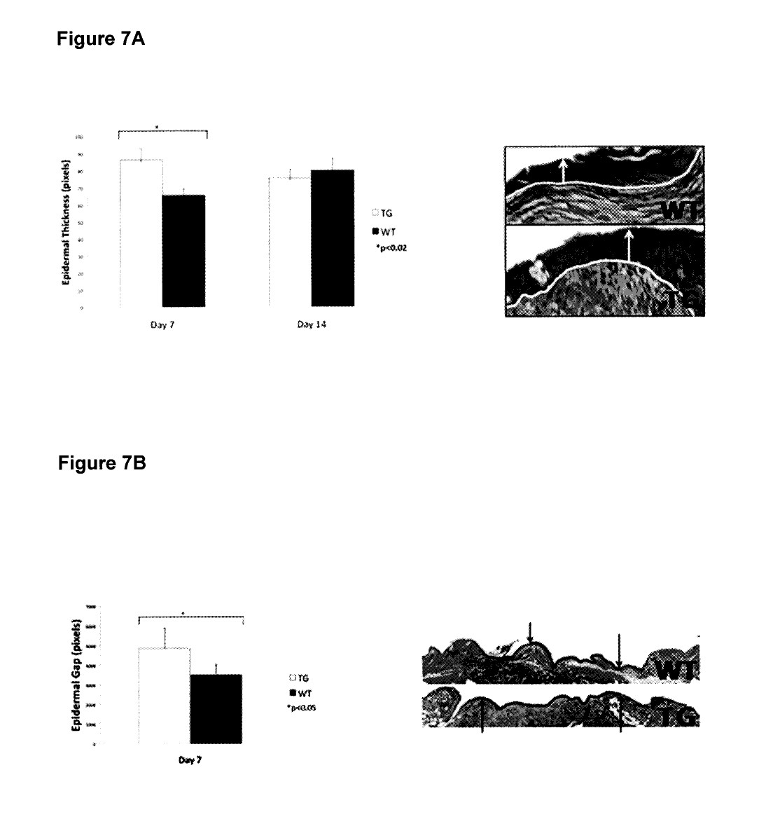

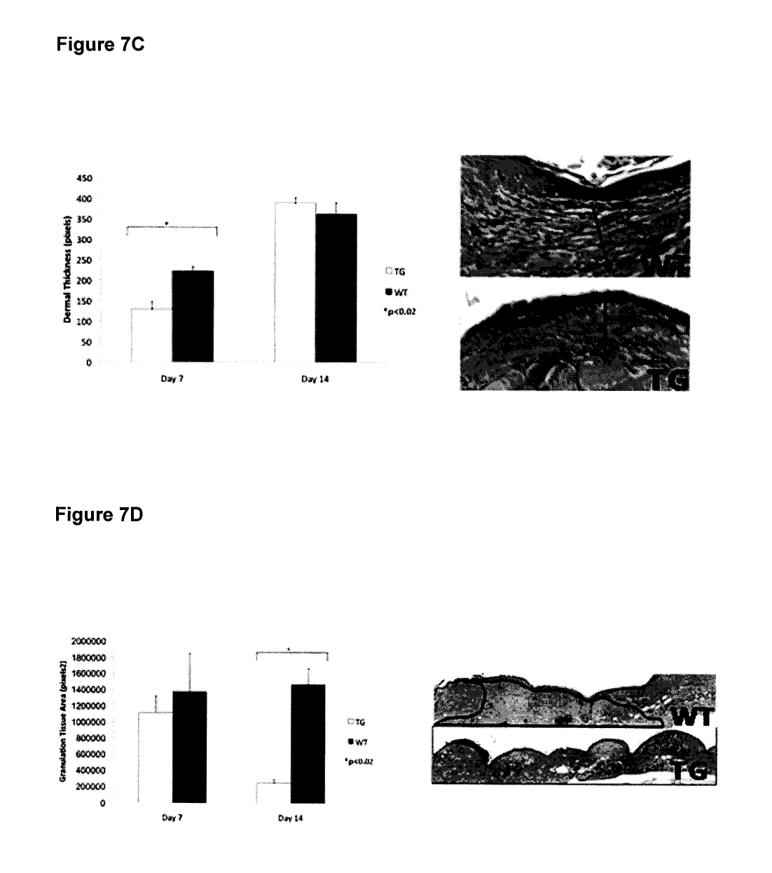

Epidermal thickness is an indicator of keratinocyte proliferation at the wound edge and indicates how the epidermis grows to form a barrier over the skin. TGF-β is a potent inhibitor of keratinocyte proliferation. As shown in FIG. 7A, CD109 transgenic mice display increased dermal thickness at 7-days post-wounding as compared to wild-type littermates suggesting that CD109 promotes keratinocyte proliferation during the first 7 days of wound healing. These results are consistent with an inhibitory effect of CD109 on the growth inhibitory effect of TGF-β on these cells. No detectable differences in epidermal thickness of transgenic and wild-type wounds are noted at 14-days post-wounding. These data suggest that CD109 accelerates the early stages of the wound healing response. The exact nature of the effects of CD109 on the kinetics of wound healing acceleration requires further studies.

Another interesting finding of this study is that excisional wounds in CD109 transgenic mice show an increased epidermal gap as compared to wild-type mice at 7-days post-wounding, suggesting that CD109 inhibits keratinocyte migration (FIG. 7B). However, at 14-day post-wounding, the wounds in both the transgenic and wild-type mice had completely re-epithelialized suggesting that the CD109 effect on migration is transient or moderate. On the other hand, it is possible that the decreased migration is compensated by the increased proliferation (epidermal thickening) and might account for the lack of delay in wound closure in transgenic mice.

In addition, excisional wounds of CD109 transgenic mice display reduced dermal thickness compared to wild-type controls at day 7, but this difference becomes undetectable by day 14 (FIG. 7C). The decreased dermal thickening of the skin in CD109 transgenic mice is consistent with the decreased cellularity and more organized collagen deposition in these mice. These results support the notion that CD109 has anti-scarring potential. An important aspect of the wound healing process is the presence of granulation tissue comprised of fibroblasts, blood vessels, ECM which facilitate the restoration of the integrity of the skin (Brandstedt et al., Acta Chir Scand, 1980. 146(8): 545-9; Midwood et al., J Investig Dermatol Symp Proc, 2006. 11(1): 73-8).

The area of the granulation tissue at 7 and 14 days post-wounding in excisional wounds of transgenic and wild-type mice was assessed and it was found that there was little difference at 7 days but significantly less granulation tissue at 14 days post-wounding in the CD109 transgenic mice, suggesting that CD109 promotes resolution of granulation tissue (FIG. 7D). These results support the contention that CD109 has anti-scarring activity.

CD109 decreases immune cell numbers in excisional wounds: Inflammation is the first phase of wound healing and plays an important role in eliminating pathogens from the site of injury and releasing growth factors for neighboring cells to initiate wound closure. Inflammation occurs immediately after platelets degranulate at the fibrin clot, first recruiting neutrophils to the wound site and soon after attracting macrophages as well. Despite the importance of inflammation during wound healing, excessive inflammation or inflammatory cell recruitment has been associated with increased scarring (Stramer et al., J Invest Dermatol, 2007. 127(5): 1009-17).

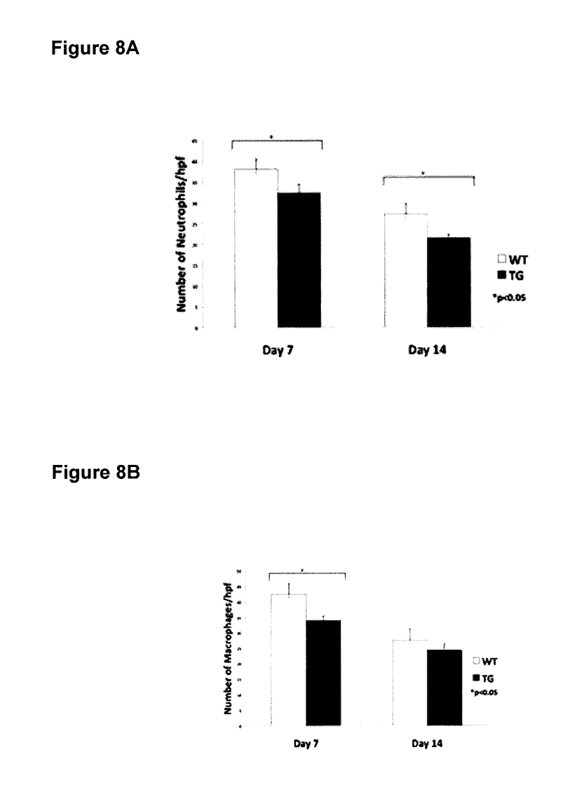

Macrophage and neutrophil content were analyzed in excisional wounds by immunohistochemistry using antibodies against the cellular markers F4/80 and Ly6-GR, respectively (Wang, X., et al., Proc Natl Acad Sci USA, 1997. 94(1): 219-26). As shown in FIGS. 8A and 8B, excisional wounds in CD109 transgenic mice have significantly decreased numbers of macrophages (FIG. 8B) and neutrophils (FIG. 8A) as compared to wild-type littermate wounds on both days 7 and 14 post-wounding. TGF-β acts as a strong chemoattractant and CD109 might antagonize this property to reduce the recruitment of inflammatory cells to the wound site. Since diminished immune cell presence and inflammation are associated with decreased scarring, these data support CD109 anti-scarring properties.

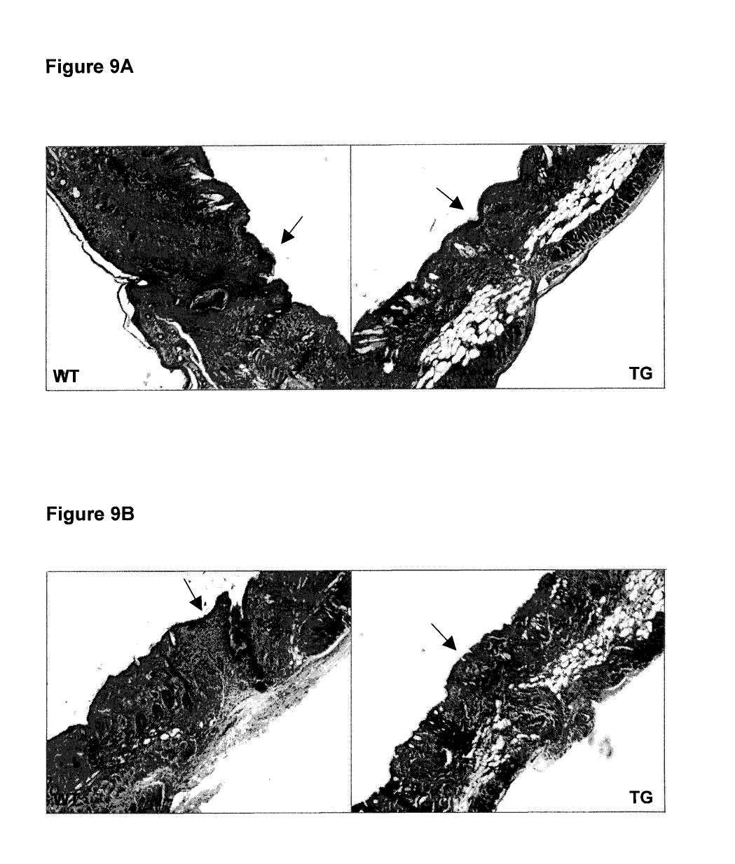

CD109 reduces cellularity and promotes a smoother epidermis in incisional wounds: Histological analysis of incisional wounds in CD109 transgenic mice show improved wound healing than incisional wounds in wild-type littermates. On 7 day post-wounding (FIG. 9A), decreased dermal cellularity and granulation tissue and a smoother epidermis were detected in the incisional wounds of CD109 transgenic mice as compared to wild-type littermate wounds. Similar results were obtained on Day 14 (FIG. 9B). In addition, these data are in agreement with the data obtained in excisional wound in the CD109 transgenic mice. Together these results underscore the anti-scarring potential of CD109.

### Discussion

The current study shows that overexpression of CD109 in the epidermis reduces scarring in murine excisional and incisional wound models. It demonstrates that that excisional wounds in CD109 transgenic mice display improved wound healing as detected by more organized collagen assembly, increased keratinocyte proliferation and epidermal thickening, as compared to those in wild type littermates. Furthermore, excisional wounds in CD109 transgenic mice exhibit reduced scarring parameters as evidenced by decreased dermal cell cellularity, decreased dermal thickening, decreased inflammatory cell number and increased granulation tissue resolution. No detectable difference in wound closure was observed between CD109 transgenic mice and their wild type littermates.

Results obtained in incisional wounds in CD109 transgenic mice support the results obtained in excisional wounds in CD109 transgenic mice. Incisional wounds in CD109 transgenic mice display decreased dermal cellularity, reduced granulation tissue, improved collagen organization and a smoother epidermis on day 7 and day 14 post wounding.

CD109 may exert its effects on wound healing by dampening TGF-β mediated processes such as keratinocyte growth inhibition, fibroblast activation, and immune cell recruitment.

In conclusion, these findings suggest that CD109 reduces scarring without impairing wound healing in vivo in a mouse model and support its therapeutic value as an anti-scarring agent.

### Example 3

### Efficacy of Recombinant CD109 Protein and a CD109-Based Peptide as Anti-Scarring Agents

To explore the potential of CD109 as a TGF-β1 antagonist, the efficacy of recombinant CD109 protein and a CD109 peptide based on the putative TGF-β binding region was examined for their ability to bind TGF-β1 and inhibit TGF-β1 signaling in vitro. The results demonstrate that it may be possible to specifically manipulate the action of TGF-β1 and the TGF-β1/β3 isoform ratios, using the full length protein and the CD109 peptide based on the putative TGF-β binding region. This suggests these proteins may be of use as therapeutic anti-scarring agents.

### Experimental

Affinity labeling assay: CD109 protein and peptides were tested for their ability to compete with ^{I125}TGF-β1 for the TGF-β signaling receptors in an affinity labeling assay. HaCaT cells were incubated with ^{I125}TGF-β1 in the presence or absence of soluble CD109 protein and CD109 peptides. Proteins from the cell membrane extracts were then separated by 3%-11% SDS-PAGE and visualized by autoradiography/phosphorimager analysis.

Western blot analysis: For Western blot studies, HaCaT cells were transfected with CD109 or EV, 24 h after transient transfection, HaCaT cells were incubated for 18 h (for the determination of fibronectin and PAI-1 levels) with the indicated amounts of TGF-31 under serum-free conditions. Cell lysates were prepared and analyzed by Western blot using the indicated antibodies.

Surface plasmon resonance assay: CD109 protein and peptides were tested for their ability to bind TGF-β using surface plasmon resonance. CD109 protein and peptides were amine-coupled onto a gold-dextran CM5 sensor chip as the ligands and TGF-β1/2/3 in HBS-ET buffer as the analytes were pumped across the CD109 amine-coupled sensor chip and binding response was measured using BIACORE 3000™ SPR detection system.

CAGA₁₂-lux assay: HaCaT cells were co-transfected CAGA₁₂-lux reporter constructs, and with β-galactosidase to monitor transfection efficiency. Cells were then allowed to recover for 24 h, and then incubated for 16 h in serum-free media containing 50 nM peptide A or GST control with 0-100 pM of TGF-beta1. Cell lysates were prepared, analyzed for luciferase activity, and the values were normalized to beta-galactosidase activity.

### Results

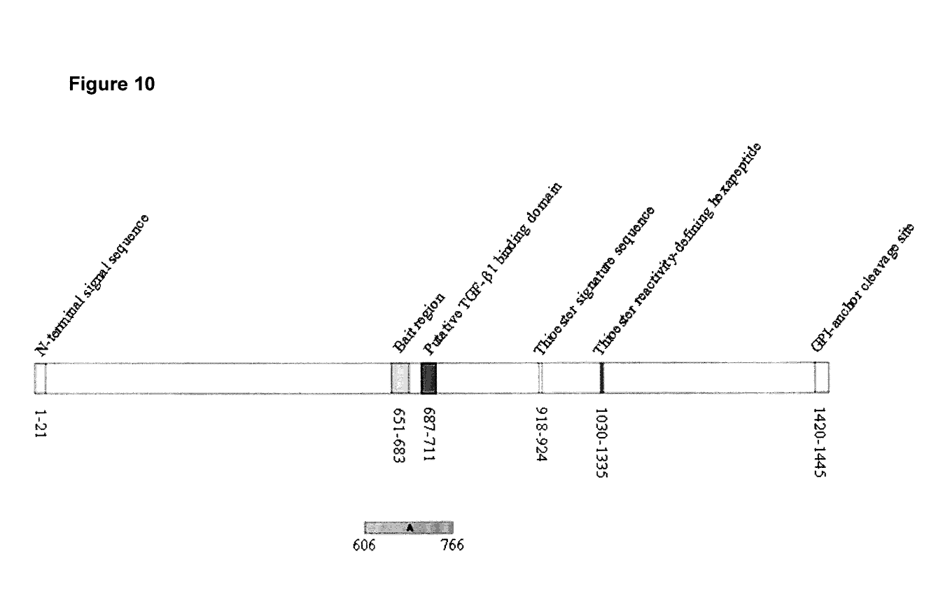

Mapping of CD109 protein: The inventors' lab has discovered a novel TGF-β1 binding protein, previously designated r150 (Tam et al. 1998) on keratinocyte skin cells. r150 was shown to bind TGF-β1, form a heteromeric complex with the TGF-β signaling receptors and inhibit downstream TGF-β induced responses (Tam et al. 2003; Finnson et al. 2006). Molecular cloning and subsequent sequencing was performed and r150 was identified as CD109 (Finnson et al. 2006), a member of the α2-M/complement superfamily expressed on endothelial cells, platelets, activated T-cells, and a variety of tumors (Solomon et al. 2004). CD109 is a membrane bound glycophosphotidyl-inositol (GPI)-anchored protein that shares several structural features with the α2-M family, including a putative bait region, thioester signature sequence and a putative TGF-β1 binding region (FIG. 10). TGF-β1 binding region on human α2-M has been mapped to a 16 amino acid region (Webb et al. 2000) that, through sequence analysis, does share some similarity with CD109. Specifically, acidic residues Glu714 and Asp719 (numbering based on α2-M) which may confer similar functionality to residues on TβRII responsible for its binding affinity for TGF-β ligand (Arandjelovic et al. 2003) are conserved on α2-M and CD109. Based on the sequence alignment analysis of α2-M and CD109, a 160 amino acid peptide (CD109 Peptide A-CD109 amino acid number 606 to 766) was constructed based around the putative TGF-β1 binding region of CD109 (FIG. 10).

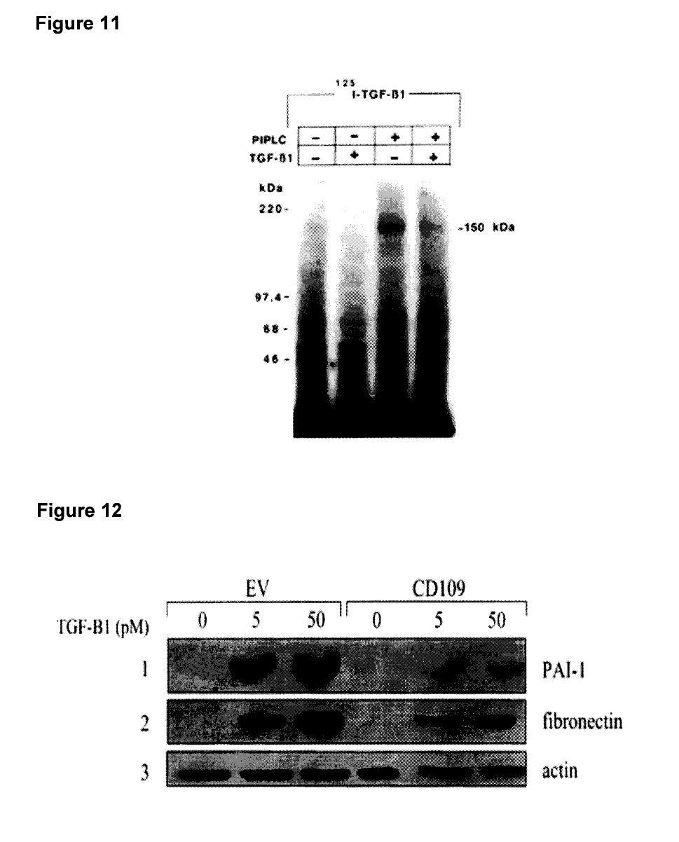

Soluble endogenous CD109 binds TGF-β1 and inhibits TGF-β1 binding to its signaling receptors: Affinity labeling assay: To verify that the soluble form of CD109 can bind to TGF-β1, human keratinocytes (HaCaT) were left untreated or treated with 0.6 U/ml of PIPLC. The GPI-anchored proteins released into the supernatant were concentrated and an aliquot was affinity cross-link labeled with 150 pM of ^{I125}TGF-β1 in the absence or presence of excess unlabeled TGF-β1 and subjected to SDS-PAGE under reducing conditions (FIG. 11). Soluble CD109 in the supernatant obtained from keratinocytes treated with PIPLC enzyme could be affinity labeled with ^{I125}TGF-β1. This binding was specific since it was markedly reduced when the labeling was done in the presence of unlabeled TGF-β1. The fact that released CD109 binds TGF-β1 indicates that CD109 is capable of binding the ligand in the absence of type I, II, and III TGF-β receptors or an intact membrane structure. Furthermore, the released CD109 can inhibit TGF-β binding to its receptors (Tam et al. 2001).

CD109 inhibits ECM synthesis: Western blot analysis of PAI-1 and Fibronectin Expression: TGF-β1 is a key regulator of ECM synthesis and breakdown, thus we examined whether CD109 can regulate TGF-β1-induced plasminogen activator inhibitor-1 (PAI-1) or fibronectin expression. FIG. 12 demonstrates that empty vector (EV) transfected HaCaT cells show a robust dose-dependent increase in PAI-1 and fibronectin protein expression in response to TGF-β1 treatment (FIG. 12, upper and middle panels, respectively). FIG. 12 demonstrates that CD109 transfected HaCaT cells display an approximate 2-fold and 7-fold decrease in PAI-1 levels at 5 pM and 50 pM TGF-β1, respectively (FIG. 12, upper panel), and 2- and 3-fold decreases in fibronectin levels at 5 pM and 50 pM TGF-β1, respectively (FIG. 12, middle panel) as compared to empty vector (EV) transfectants. Reprobing the membrane with an anti-actin Ab demonstrates that equivalent amounts of protein were loaded in each lane (FIG. 12, bottom panel).

Surface Plasmon Resonance shows that full-length CD109 and a CD109-based 160 amino acid peptide (Peptide A) bind TGF-β1 with high affinity: To determine whether the CD109 protein and CD109 peptide based around the putative TGF-β binding region (CD109 Peptide A) would bind directly to TGF-β, surface plasmon resonance (SPR) analysis was performed using soluble CD109 protein and CD109 Peptide A. SPR results revealed that soluble CD109 protein binds TGF-β1 with high affinity (KD=1×10⁻¹⁰ M), TGF-β2 with moderate affinity (KD=1×10⁻⁹ M) and TGF-β3 with no low affinity (data not shown). SPR results also revealed that CD109 Peptide A binds TGF-β1 with moderate affinity (KD=1×10⁻⁸M) (data not shown). Collectively, SPR results show CD109 protein and CD109 Peptide A bind directly to TGF-β.

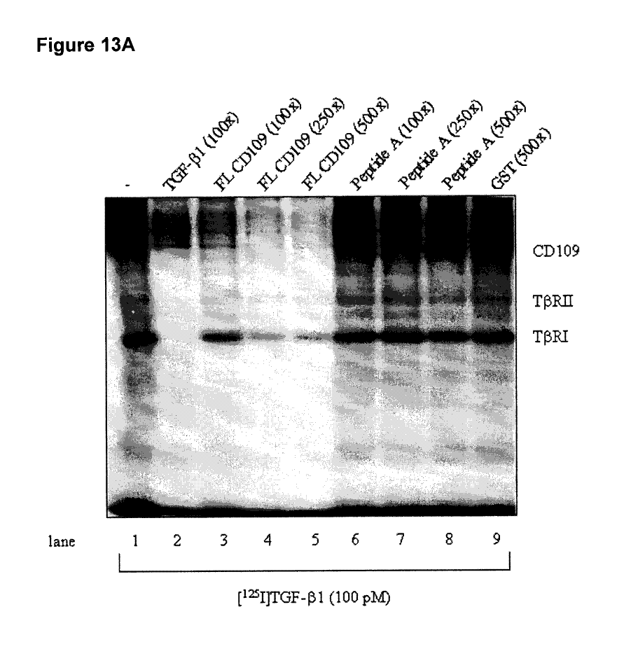

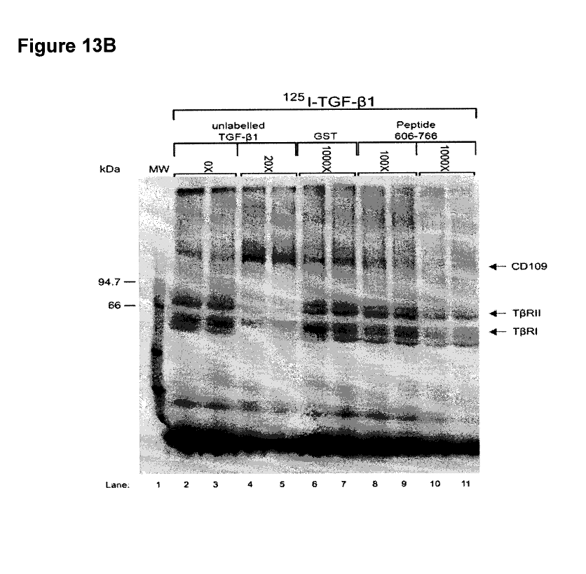

Recombinant full-length CD109 and Peptide both inhibit TGF-β binding to types I and II TGF-β receptors: Affinity labeling assay. Affinity labeling of keratinocytes with ^{I125}TGF-β1 was performed following CD109 protein and CD109 peptide treatment to determine whether CD109 soluble full-length protein (FL CD109) and CD109 peptide based around the putative TGF-β binding region (CD109 Peptide A) are able to inhibit ^{I125}TGF-β1 binding to its cell surface receptors. Our results show that both FL CD109 protein and CD109 Peptide A are able to inhibit ^{I125}TGF-β1 binding in a dose-dependent manner to its cell surface receptors by directly binding ^{I125}TGF-β1 and sequestering it away from its cell surface receptors (FIGS. 13A and 13B).

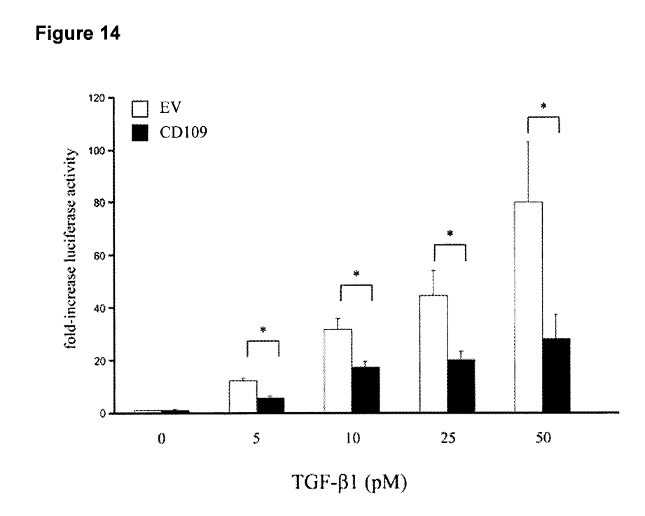

FIG. 14 shows that CD109 inhibits TGF-□-induced transcriptional activity in human keratinocytes. HaCaT cells were transfected with a TGF-β responsive luciferase reporter construct (CAGA₁₂-lux) and β-galactosidase to measure transfection efficiency. Transfected cells were treated for 18 hours without or with 0-50 pM TGF-β1. Cell lysates were prepared and analyzed for luciferase and β-galactosidase activities and data are presented as luciferase activity (luciferase/β-galactosidase).

### Conclusions

These results indicate that soluble CD109 protein and CD109 peptide corresponding to the putative TGF-β1 binding region of CD109 (CD109 Peptide A) inhibit TGF-β1 binding to its receptors and TGF-β signaling in vitro. These results also show that CD109 binds to TGF-β1 with high affinity, TGF-β2 with moderate affinity and TGF-β3 with low affinity. The TGF-β isoform specific binding nature of CD109 supports its potential as an anti-scarring agent. Together, these results demonstrate that full length recombinant CD109 and a CD109-based peptide (Peptide A) bind TGF-β1 with high affinity, inhibit TGF-β1 binding to its receptors and decrease TGF-β signaling. These findings provide a basis for a novel therapeutic approach in which a novel CD109 peptide may be used to modulate TGF-β action locally to therapeutically treat atypical scarring, and may also have relevance to diseases such as scleroderma and psoriasis where dysregulation of TGF-β action is implicated.

### REFERENCES

Arandjelovic, S., T. A. Freed, and S. L. Gonias, Growth factor-binding sequence in human alpha2-macroglobulin targets the receptor-binding site in transforming growth factor-beta. Biochemistry, 2003. 42(20): p. 6121-7.
Blobe, G. C., Schiemann, and W. P., Lodish, H. F., Role of Transforming Growth Factor β in Human Disease. N Engl J Med, 2000. 342(18): p. 1350-1358.
Finnson, K. et al., Identification of CD109 as part of the TGF-β receptor system in human keratinocytes. FASEB Journal, 2006. 20(9): p. 1525-7.
O'Kane, S, and Ferguson M. W. J., Transforming Growth Factor β and Wound Healing. The International Journal of Biochemistry & Cell Biology, 1997. 29(1): p. 63-78.
Solomon, K. R., et al., CD109 represents a novel branch of the alpha2-macroglobulin/complement gene family. Gene, 2004. 327(2): p. 171-83.
Tam, B. et al., TGF-β receptor expression on human keratinocytes: A 150 kDa GPI-anchored TGF-β1 binding protein forms a heterometric complex with type I and type II receptors. Journal of Cellular Biochemistry, 1998. 70: p. 573-586.
Tam, B. et al., Characterization of a 150 kDa accessory receptor for TGF-β1 on keratinocytes: direct evidence for a GPI anchor and ligand binding of the released form. Cell Biochem, 2001. 83(3): p. 494-507.
Tam, B. et al., Glycosylphosphatidylinositol-anchored proteins regulate transforming growth factor β signaling in human keratinocytes. Journal of Biological Chemistry, 2003. 278(49): p. 49610-49617.
Webb, D. J., et al., A 16-amino acid peptide from human alpha2-macroglobulin binds transforming growth factor-beta and platelet-derived growth factor-BB. Protein Sci, 2000. 9(10): p. 1986-92.

### Example 4

### CD109 Regulates Keratinocyte Phenotype in Psoriasis

Psoriasis is a chronic inflammatory skin disorder characterized by epidermal keratinocyte hyperproliferation, leukocyte infiltration and alterations in cytokine production. The current example documents that CD109 protein expression is markedly decreased in psoriatic skin although CD109 mRNA levels remain unchanged. Release of CD109 from keratinocyte cell surface by PI-PLC or treatment with recombinant CD109 protein results in the downregulation of type I TGFβ receptor and decreased TGFβ/Smad signaling. This signaling change is associated with increased proliferation, upregulation of STAT3, phospho-STAT3 and Bcl-2 expression in keratinocytes. Taken together, these findings suggest that CD109 regulates keratinocyte phenotype in psoriasis.

### Materials and Methods

Cell Culture: HaCaT cells were obtained from Dr. Fusening, German Cancer Research Center, Heidelberg (Boukamp et al. 1988), while N/TERT-1 and NE6E7 lines were generously provided by Dr. J Rheinwald (Harvard Medical School, Boston, Mass.) (Dickson et al. 2000). HaCaT-GPI mutated line was previously derived in our laboratory and was described elsewhere (Tam et al. 2003). HaCaT and HaCaT-GPI mutated cells were serially passaged in DMEM media (Invitrogen Life Technologies, Carlsbad Calif.) containing 10% fetal bovine serum (FBS) (Invitrogen Life Technologies). The N/TERT-1 and NE6E7 cells were grown in Keratinocyte Serum-Free Defined Media supplemented with Bovine Pituitary Extract (BPE) and recombinant EGF (Invitrogen Life Technologies). All cells were grown in 5% CO₂, 95% air humidified incubator at 37° C. For Western Blot and growth curve analyses cells were plated into 6 well plates or T25 flasks and were treated with 0.5 units/mL of PI-PLC (Sigma, St. Louis, Mo.), 400 ng/mL of CD109 recombinant protein (R&D Systems, Burlington, ON) or TGFβ1 (Genzyme, Framingham, Mass.) at 5, 50 or 100 pM concentrations.

Growth Assays: Cells were plated in flasks at equal numbers in the presence or absence of a TGFβ peptide +/−CD109 recombinant protein and were then allowed to grow for a set number of days and then counted manually at various time points. N/TERT-1 cells were also subjected to clonigenic growth assay as previously described (Guda et al. 2007) in the presence or absence of 400 ng/mL of CD109 recombinant protein.

Real-Time TaqMan™ RT-PCR Quantitation CD109 Expression: Total RNA was extracted with Qiagene RNeasy™ Mini-Kit (Valencia, Calif.) according to the manufacturer's instructions. The RT-PCR was carried out on the BioRad ICycler™ (Hercules, Calif.) using the iCycler™ SYBR kits with appropriate primers as previously described (Litvinov et al. 2006; Finnson et al. 2006).

siRNA knockdown of CD109 expression: siRNA specific to CD109 and control siRNA were purchased from Ambion (Austin, Tex.) and transfected into N/TERT-1 cells via Lipofectamine 2000™ reagent (Invitrogen Life Technologies) according to the manufacturer's instructions. Subsequently cells were lysed and analyzed by a Western Blot.

Western Blotting: Each lane contained whole-cell lysates collected from 10⁵ cells. Lysates were fractionated on 7.5% SDS-PAGE gels and subsequently transferred onto PVDF membranes (Bio-Rad Laboratories). Western blotting was performed as described previously (Litvinov et al. 2006) using the appropriate antibodies. Specifically, rabbit polyclonal STAT3, rabbit polyclonal β-actin and rabbit polyclonal pSmad 2/3 antibodies were purchased from Santa Cruz Biotechnology (Santa Cruz, Calif.). CD109 mouse monoclonal antibody was purchased from BD Pharmingen (Hunt Valley, Md.). Phospho-STAT3 (Tyr 705), TGF-β Receptor I and Bcl-2 rabbit polyclonal antibodies were purchased from Cell Signaling (Danvers, Mass.). All secondary horseradish peroxidase-conjugated antibodies and chemiluminescent detection reagents (ECL) were purchased from Amersham Biosciences (Piscataway, N.J.).

Tissue Collection and Immunohistochemistry: 3 mm diameter skin punch biopsies were obtained from psoriatic patients and healthy individuals at the Montreal General Hospital Dermatology Clinic after receiving their informed consent in accordance to the McGill University Health Center IRB Approved Study Protocols #01-054 and #03-050. Patients were 25-67 year old males and females. All psoriatic patients (PASI scores 2-4) were undergoing ultraviolet light B (UVB) phototherapy 2-3 days per week and received no other systemic treatments. Tissues were collected before their sessions of UVB treatment. Obtained punch skin biopsies were snap frozen immediately in liquid nitrogen. All tissues were cut with the cryostat and tissue slides were fixed in 80% methanol solution for 30 minutes and then subjected to immunohistochemistry analysis as previously described elsewhere (Litvinov et al. 2006) utilizing the same reagents as for the immunocytochemistry assay and the same antibodies as for the western blotting.

### Results and Discussion

Cell Culture Models to Study CD109 function in Skin: N/TERT-1 and NE6E7 cells are immortalized, non-transformed human keratinocytes that were obtained via hTERT transfection or HPV virus infection respectively and are routinely used to study skin diseases (Dickson et al. 2000). In addition, a spontaneously immortalized HaCaT keratinocyte line (Boukamp et al. 1988) was chosen to corroborate the findings obtained in N/TERT-1 and NE6E7 cells.

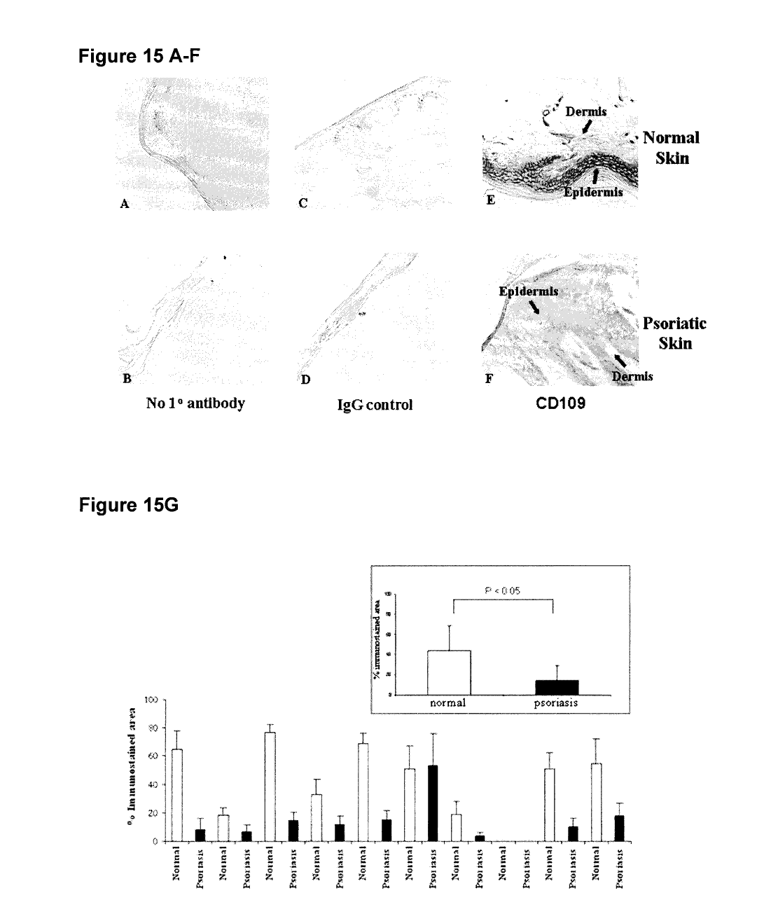

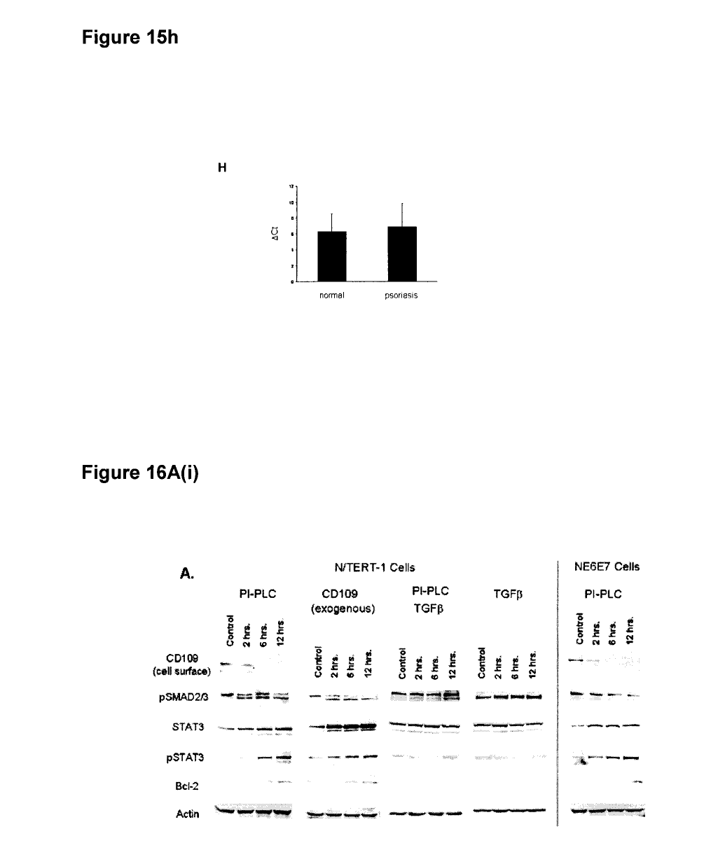

CD109 Protein, but not mRNA, is reduced in the Psoriatic Epidermis: CD109 protein expression was determined in normal and psoriatic skin by immunohistochemical analysis. The results in FIG. 15A-F show that the expression of CD109 protein is decreased in psoriatic epidermis in comparison to normal skin. The immunostaining intensity was determined by measuring the area of each skin section pictures at two distinct and fixed thresholds intensity and by calculating the average ratio of the area values obtained using ImagePro™, as described in the Methods. The results are represented quantitatively by bar graphs and illustrate the decrease of CD109 expression pattern in psoriatic skin compared to adjacent normal one in 8 out of 10 patients (FIG. 15G). To determine whether the observed decrease in CD109 expression also occurs at the mRNA level, real-time RT-PCR with CD109 specific primers was performed on total RNA isolated from the epidermis of normal and psoriatic skin. These results document high levels of CD109 mRNA in normal and psoriatic epidermis with no significant differences between samples (FIG. 15H). Thus, while CD109 mRNA is strongly expressed in normal and psoriatic skin the CD109 protein is decreased in the psoriatic epidermis.

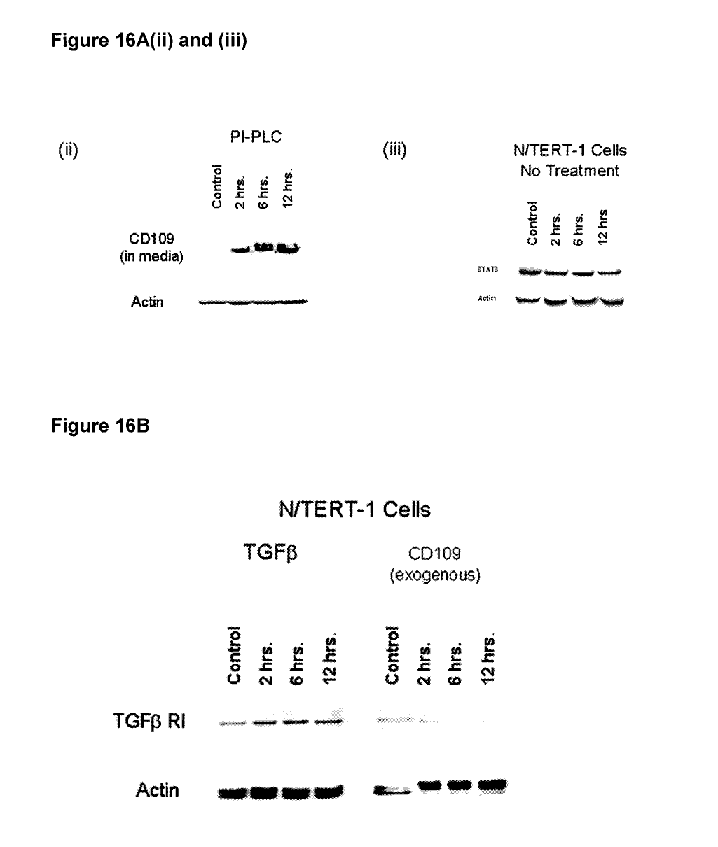

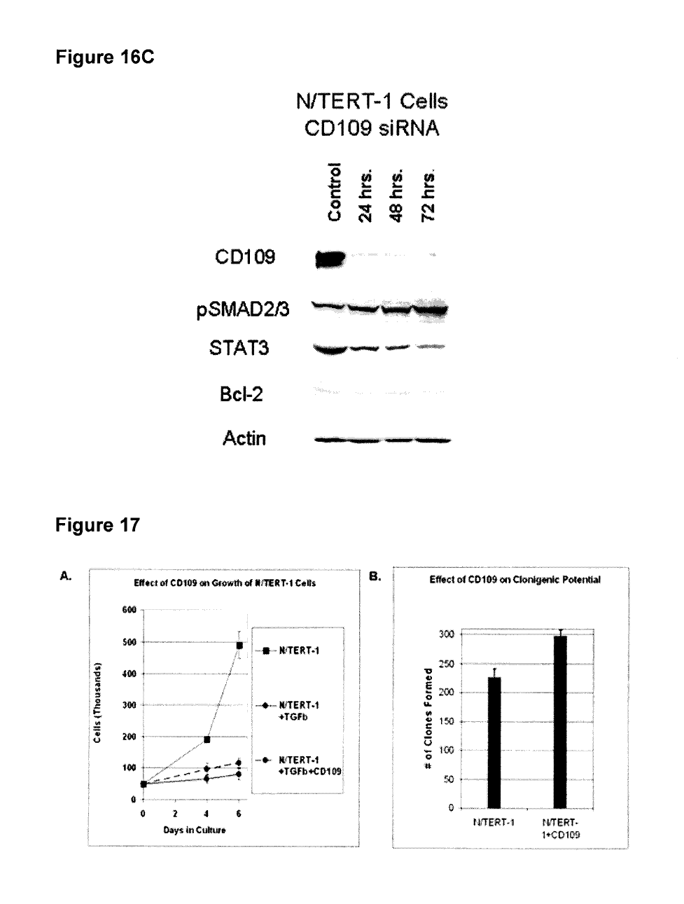

Release of CD109 from the Cell Surface or Addition of Recombinant CD109 Protein Downregulates TGFβ/Smad Signaling and Upregulates STAT3 Expression and STAT3 Phosphorylation in Human Keratinocytes: CD109 is a GPI-anchored protein and is released from the cell surface into the extracellular milieu, where it may interact with the TGFβ protein and TGFβ receptor in a similar way as the membrane bound CD109 (Finnson et al. 2006). Such release of CD109 can be effectively achieved by treating N/TERT-1 and NE6E7 keratinocyte cells with PI-PLC for 0 (i.e., control), 2, 6, and 12 hours. As documented by a Western blot, PI-PLC treatment releases CD109 protein into the media (FIG. 16A) and results in a loss of CD109 cell surface protein expression in N/TERT-1 and NE6E7 keratinocytes (FIG. 16A). Such CD109 release subsequently alters TGFβ signaling as manifested by downregulation in SMAD2 phosphorylation in N/TERT-1 and NE6E7 cells (FIG. 16A). Importantly, such phospho-SMAD2 downregulation results in upregulation of STAT3 expression and phosphorylation (FIG. 16A). In the absence of PI-PLC or other treatment STAT3 expression over time remains unchanged (FIG. 16A). Upregulation of STAT3 has been previously recognized as a critical hallmark of keratinocyte activation in response to injury and in psoriasis (Sano et al. 2005a; Sano et al. 2005b, Sano et al. 2008). To further examine the other signaling changes, the expression of Bcl-2 antiapoptotic protein was evaluated which, as documented in FIG. 16A, is also upregulated at 6-12 hours after PI-PLC treatment. Such finding is consistent with previous observation that the expression of Bcl-2 family anti-apoptotic genes (i.e., Bcl-2 and Bcl-x_{L}) is under a direct transcriptional regulation of STAT3 (Alvarez et al. 2004; Grad et al. 2000; Hodge et al. 2005). To determine whether these effects might be attributed to the released CD109, experiments were carried out to examine whether recombinant CD109 protein mimicked these effects. As documented by a western blot, exogenous CD109 treatment produces the same responses as the PI-PLC treatment (FIG. 16A), thereby suggesting that the previously observed effects of PI-PLC treatment are likely attributed to the released CD109 from the cell surface.

As previously discussed, the documented CD109 inhibition of the TGFβ signaling may occur via 1) binding and sequestration of the TGFβ ligand and/or 2) binding and destabilization of the TGFβ receptor protein (Finnson et al. 2006). Thus, we tested whether addition of the exogenous TGFβ protein can overcome the CD109 sequestration of the TGFβ signal and subsequent downregulation of the TGFβ signaling. As demonstrated in FIG. 16A, PI-PLC treatment supplemented with 50 pM of exogenous TGFβ protein significantly reduces the PI-PLC induced (i.e., CD109-mediated) STAT3 phosphorylation and Bcl-2 upregulation, but failed to neutralize these effects completely as compared to the 5 pM TGFβ treatment alone (FIG. 16A). These findings support previous observations that CD109 sequesters TGFβ signaling, but also suggest that CD109 may downregulate TGF signaling at the level of the receptor. To support this hypothesis, the expression of the TGFβ RI levels was analyzed in N/TERT-1 cells treated with CD109 recombinant protein (FIG. 16B). Such supplementation of media with CD109 recombinant protein resulted in a significant downregulation of the TGFβ RI protein (FIG. 16B).

CD109 siRNA Treatment of Human Keratinocytes Results in Downregulation of STAT3 Protein Expression. While the release and activation of CD109 via PI-PLC treatment results in a downregulation of the TGFβ signaling and upregulation of STAT3 expression, whether normal human N/TERT-1 keratinocytes exhibit baseline activity of CD109 without exogenous PI-PLC was questioned. To block CD109 activity the siRNA strategy was employed and the subsequent changes in TGFβ signaling, STAT3 and Bcl-2 expression was studied (FIG. 16C). The CD109 specific siRNA was able to dramatically downregulate the cellular levels of CD109 protein and to produce an increase in TGFβ signaling as documented by the upregulation in phospho-SMAD2. Furthermore, elimination of CD109 activity resulted in downregulation of STAT3 expression and did not affect the Bcl-2 expression. These findings further implicate released CD109 as an important regulator of TGFβ and STAT3 signaling.

Recombinant CD109 Protein Increases Cell Growth and Survival: Next the effect of the released CD109 on keratinocyte growth was examined. Cells were grown in the media supplemented with 5 pM of TGFβ in the presence or absence of CD109 exogenous protein (FIG. 17A). As documented by the growth assays, CD109 treatment modestly alleviated the TGFβ-mediated suppression of cell growth (FIG. 17A). These findings are consistent previous reports indicating an increased proliferation and decreased apoptosis of keratinocytes, when TGFβ signaling is inhibited (Amendt et al 2002). To further evaluate the impact of CD109 function and the documented Bcl-2 upregulation on cell survival, a clonigenic assay was carried out in the presence or absence of CD109 treatment (FIG. 17B), where five hundred N/TERT-1 cells were seeded in 10 cm tissue culture dishes and the produced colonies were subsequently counted 3 weeks later. The ability of cells to form a colony is a direct manifestation of cell survival. As documented in FIG. 17B, there was an increased survival (i.e., colony formation) in the presence of CD109 thereby suggesting that the observed CD109-mediated upregulation of Bcl-2 may be important for keratinocyte survival.

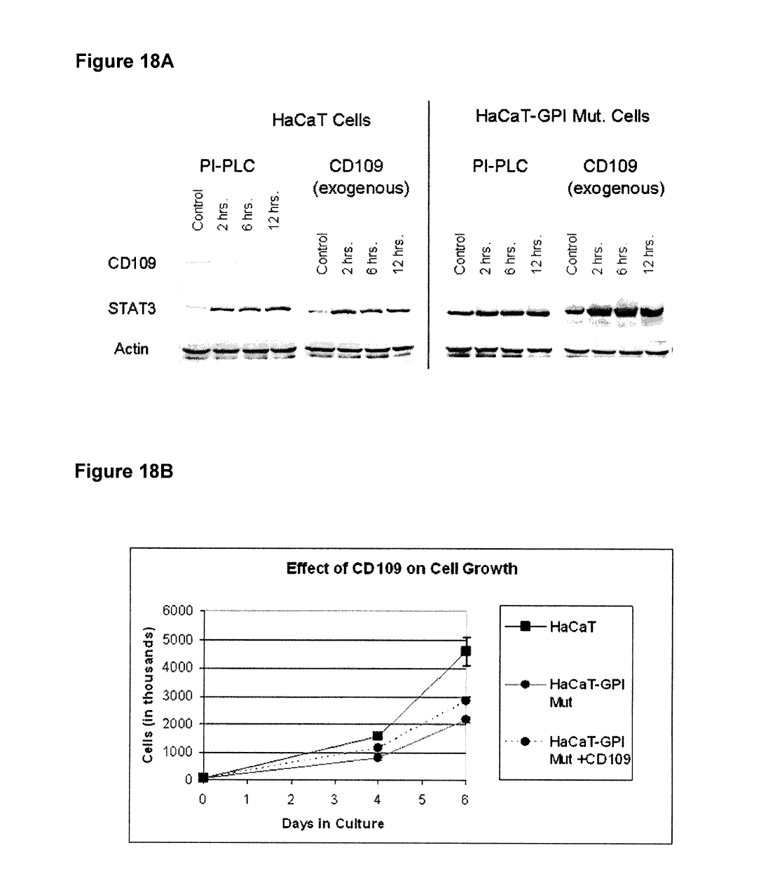

Release of CD109 from the Cell Surface or Addition of CD109 Protein Upregulates STAT3 Expression and Enhances Proliferation in HaCaT cells: HaCaT cells are one of the most widely used keratinocyte cell lines to study skin diseases and processes and provide an additional cell culture model to confirm our observations in N/TERT-1 and N/E6E7 cells. As demonstrated in FIG. 18A (left panel) HaCaT cells treated with PI-PLC upregulate the expression of STAT3. This effect was reproduced when cells were also treated with the exogenous CD109 protein.

A HaCaT mutant line defective in GPI-anchor biosynthesis (HaCaT-GPI Mutant) that shows reduced expression of CD109 on the cell surface has been established previously (Finnson et al 2006; Tam et al. 2003). Analysis of this line reveals that PI-PLC treatment has no effect in these cells as documented by a lack of STAT3 upregulation (FIG. 18A right panel). These findings are consistent with the fact that the HaCaT-GPI Mutant cells do not express the CD109 protein on the cell surface and, therefore, PI-PCL treatment is not able to alter the expression of STAT3 protein. However, when these cells are treated with the exogenous CD109 protein they respond similarly to the parental HaCaT cells and upregulate STAT3 expression (FIG. 18A, left panel). Subsequently, in carrying out a cell growth assay on HaCaT vs. HaCaT-GPI Mutant cells it was noted that the mutant cells grow slower then their parental counterparts (FIG. 18B). Such finding could be explained by the enhanced TGFβ signaling in the mutant cells due to the lack of CD109 inhibition. Interestingly, in the HaCaT GPI-Mutant cells similarly to the finding in the N/TERT-1 cells, the addition of exogenous CD109 protein modestly improves cell growth by downregulating the levels of the TGF signaling (FIG. 16B and (Finnson et al. 2006)). These data confirm previous results obtained in N/TERT-1 and NE6E7 cells and further indicate that human keratinocytes are acutely sensitive to the effects of CD109 function.

The results shown in FIG. 15A-F and FIG. 15G further demonstrate that in 8 out of 10 psoriasis patients CD109 protein levels are markedly lower in the lesional skin as compared to normal skin. These data suggest that CD109 expression inversely correlates with a psoriasis phenotype and suggest that CD109 can be of value as a biomarker for psoriasis.

### Conclusion

The present study demonstrates that the expression of CD109 protein is markedly decreased in lesional psoriatic skin as compared to adjacent normal skin in 8 out of 10 patients. However, CD109 mRNA levels are similar in normal and lesional skin. Release of CD109 from the cell surface of normal keratinocytes or the addition of recombinant CD109 protein results in a psoriasis-like phenotype, as detected by increased proliferation, enhanced STAT3 activation, decreased phosphoSmad2 activation and TGF-β receptor downregulation. Others have reported that GPI anchored proteins are lost form the cell surface of psoriatic keratinocytes and that this loss is due to an increased release from the cell surface rather than a decrease in their synthesis (Venneker et al. 1994). Although the current study does not allow distinguishing between these two possibilities, the data presented demonstrate that inhibiting the release of CD109 or addition of recombinant CD109 protein promotes several key biochemical properties of psoriatic keratinocytes such as increased cell proliferation and STAT3 activation. Together, these findings shows that CD109 may represent a valuable marker for psoriasis and that targeting this molecule may be useful in the treatment of psoriasis.

### REFERENCES

1. Huerta, C., Rivero, E., and Rodriguez, L. A. 2007. Incidence and risk factors for psoriasis in the general population. Arch Dermatol 143:1559-1565.
2. Wakkee, M., Thio, H. B., Prens, E. P., Sijbrands, E. J., and Neumann, H. A. 2007. Unfavorable cardiovascular risk profiles in untreated and treated psoriasis patients. Atherosclerosis 190:1-9.
3. Domm, S., Cinatl, J., and Mrowietz, U. 2008. The impact of treatment with tumour necrosis factor-alpha antagonists on the course of chronic viral infections: a review of the literature. Br J Dermatol.
4. Ghoreschi, K., Weigert, C., and Rocken, M. 2007. Immunopathogenesis and role of T cells in psoriasis. Clin Dermatol 25:574-580.
5. Lowes, M. A., Kikuchi, T., Fuentes-Duculan, J., Cardinale, I., Zaba, L. C., Haider, A. S., Bowman, E. P., and Krueger, J. G. 2008. Psoriasis Vulgaris Lesions Contain Discrete Populations of Th1 and Th17 T Cells. J Invest Dermatol.
6. Zheng, Y., Danilenko, D. M., Valdez, P., Kasman, I., Eastham-Anderson, J., Wu, J., and Ouyang, W. 2007. Interleukin-22, a T(H)17 cytokine, mediates IL-23-induced dermal inflammation and acanthosis. Nature 445:648-651.
7. Caproni, M., Antiga, E., Melani, L., Volpi, W., Del Bianco, E., and Fabbri, P. 2008. Serum Levels of IL-17 and IL-22 Are Reduced by Etanercept, but not by Acitretin, in Patients with Psoriasis: a Randomized-Controlled Trial. J Clin Immunol.
8. Kryczek, I., Bruce, A. T., Gudjonsson, J. E., Johnston, A., Aphale, A., Vatan, L., Szeliga, W., Wang, Y., Liu, Y., Welling, T. H., et al. 2008. Induction of IL-17+ T cell trafficking and development by IFN-gamma: mechanism and pathological relevance in psoriasis. J Immunol 181:4733-4741.
9. Nograles, K. E., Zaba, L. C., Guttman-Yassky, E., Fuentes-Duculan, J., Suarez-Farinas, M., Cardinale, I., Khatcherian, A., Gonzalez, J., Pierson, K. C., White, T. R., et al. 2008. Th17 cytokines interleukin (IL)-17 and IL-22 modulate distinct inflammatory and keratinocyte-response pathways. Br J Dermatol.
10. Werner, S., Krieg, T., and Smola, H. 2007. Keratinocyte-fibroblast interactions in wound healing. J Invest Dermatol 127:998-1008.
11. Nickoloff, B. J., Bonish, B. K., Marble, D. J., Schriedel, K. A., DiPietro, L. A., Gordon, K. B., and Lingen, M. W. 2006. Lessons learned from psoriatic plaques concerning mechanisms of tissue repair, remodeling, and inflammation. J Investig Dermatol Symp Proc 11:16-29.
12. Rahimi, R. A., and Leof, E. B. 2007. TGF-beta signaling: a tale of two responses. J Cell Biochem 102:593-608.
13. Reiss, M., and Sartorelli, A. C. 1987. Regulation of growth and differentiation of human keratinocytes by type beta transforming growth factor and epidermal growth factor. Cancer Res 47:6705-6709.
14. Glick, A. B., Kulkarni, A. B., Tennenbaum, T., Hennings, H., Flanders, K. C., O'Reilly, M., Sporn, M. B., Karlsson, S., and Yuspa, S. H. 1993. Loss of expression of transforming growth factor beta in skin and skin tumors is associated with hyperproliferation and a high risk for malignant conversion. Proc Natl Acad Sci USA 90:6076-6080.
15. Sellheyer, K., Bickenbach, J. R., Rothnagel, J. A., Bundman, D., Longley, M. A., Krieg, T., Roche, N. S., Roberts, A. B., and Roop, D. R. 1993. Inhibition of skin development by overexpression of transforming growth factor beta 1 in the epidermis of transgenic mice. Proc Natl Acad Sci USA 90:5237-5241.
16. Flisiak, I., Chodynicka, B., Porebski, P., and Flisiak, R. 2002. Association between psoriasis severity and transforming growth factor beta(1) and beta (2) in plasma and scales from psoriatic lesions. Cytokine 19:121-125.
17. Nockowski, P., Szepietowski, J. C., Ziarkiewicz, M., and Baran, E. 2004. Serum concentrations of transforming growth factor beta 1 in patients with psoriasis vulgaris. Acta Dermatovenerol Croat 12:2-6.
18. Doi, H., Shibata, M. A., Kiyokane, K., and Otsuki, Y. 2003. Downregulation of TGFbeta isoforms and their receptors contributes to keratinocyte hyperproliferation in psoriasis vulgaris. J Dermatol Sci 33:7-16.
19. Finnson, K. W., Tam, B. Y., Liu, K., Marcoux, A., Lepage, P., Roy, S., Bizet, A. A., and Philip, A. 2006. Identification of CD109 as part of the TGF-beta receptor system in human keratinocytes. Faseb J 20:1525-1527.
20. Tam B Y Y, Larouche, D, Germain, L, Hooper, N M, and Philip A. Characterization of a 150 kDa accessory receptor for TGF-β1 on keratinocytes: Direct evidence for a GPI-anchor, and ligand binding of the released form. J. Cell. Biochem. 83: 494-507, 2001
Tam, B. Y., Finnson, K. W., and Philip, A. 2003. Glycosylphosphatidylinositol-anchored proteins regulate transforming growth factor-beta signaling in human keratinocytes. J Biol Chem 278:49610-49617.
21. Hagiwara, S., Murakumo, Y., Sato, T., Shigetomi, T., Mitsudo, K., Tohnai, I., Ueda, M., and Takahashi, M. 2008. Up-regulation of CD109 expression is associated with carcinogenesis of the squamous epithelium of the oral cavity. Cancer Sci 99:1916-1923.
22. Boukamp, P., Petrussevska, R. T., Breitkreutz, D., Hornung, J., Markham, A., and Fusenig, N. E. 1988. Normal keratinization in a spontaneously immortalized aneuploid human keratinocyte cell line. J Cell Biol 106:761-771.
23. Dickson, M. A., Hahn, W. C., Ino, Y., Ronfard, V., Wu, J. Y., Weinberg, R. A., Louis, D. N., Li, F. P., and Rheinwald, J. G. 2000. Human keratinocytes that express hTERT and also bypass a p16(INK4a)-enforced mechanism that limits life span become immortal yet retain normal growth and differentiation characteristics. Mol Cell Biol 20:1436-1447.
24. Guda, K., Natale, L., and Markowitz, S. D. 2007. An improved method for staining cell colonies in clonogenic assays. Cytotechnology 54:85-88.
25. Litvinov, I. V., Vander Griend, D. J., Xu, Y., Antony, L., Dalrymple, S. L., and Isaacs, J. T. 2006. Low-calcium serum-free defined medium selects for growth of normal prostatic epithelial stem cells. Cancer Res 66:8598-8607.
26. Litvinov, I. V., Vander Griend, D. J., Antony, L., Dalrymple, S., De Marzo, A. M., Drake, C. G., and Isaacs, J. T. 2006. Androgen receptor as a licensing factor for DNA replication in androgen-sensitive prostate cancer cells. Proc Natl Acad Sci USA 103:15085-15090.
27. Senoo, M., Pinto, F., Crum, C. P., and McKeon, F. 2007. p63 Is essential for the proliferative potential of stem cells in stratified epithelia. Cell 129:523-536.
28. Sano, S., Chan, K. S., Carbajal, S., Clifford, J., Peavey, M., Kiguchi, K., Itami, S., Nickoloff, B. J., and DiGiovanni, J. 2005. Stat3 links activated keratinocytes and immunocytes required for development of psoriasis in a novel transgenic mouse model. Nat Med 11:43-49.
29. Sano, S., Chan, K. S., and DiGiovanni, J. 2008. Impact of Stat3 activation upon skin biology: a dichotomy of its role between homeostasis and diseases. J Dermatol Sci 50:1-14.
30. Sano, S., Chan, K. S., Kira, M., Kataoka, K., Takagi, S., Tarutani, M., Itami, S., Kiguchi, K., Yokoi, M., Sugasawa, K., et al. 2005. Signal transducer and activator of transcription 3 is a key regulator of keratinocyte survival and proliferation following UV irradiation. Cancer Res 65:5720-5729.
31. Alvarez, J. V., and Frank, D. A. 2004. Genome-wide analysis of STAT target genes: elucidating the mechanism of STAT-mediated oncogenesis. Cancer Biol Ther 3:1045-1050.
32. Grad, J. M., Zeng, X. R., and Boise, L. H. 2000. Regulation of Bcl-xL: a little bit of this and a little bit of STAT. Curr Opin Oncol 12:543-549.
33. Hodge, D. R., Hurt, E. M., and Farrar, W. L. 2005. The role of IL-6 and STAT3 in inflammation and cancer. Eur J Cancer 41:2502-2512.
34. Amendt, C., Mann, A., Schirmacher, P., and Blessing, M. 2002. Resistance of keratinocytes to TGFbeta-mediated growth restriction and apoptosis induction accelerates re-epithelialization in skin wounds. J Cell Sci 115:2189-2198.
35. Venneker, G. T., Das, P. K., Meinardi, M. M., van Marle, J., van Veen, H. A., Bos, J. D., and Asghar, S. S. 1994. Glycosylphosphatidylinositol (GPI)-anchored membrane proteins are constitutively down-regulated in psoriatic skin. J Pathol 172:189-197.
36. Leivo, T., Leivo, I., Kariniemi, A. L., Keski-Oja, J., and Virtanen, I. 1998. Down-regulation of transforming growth factor-beta receptors I and II is seen in lesional but not non-lesional psoriatic epidermis. Br J Dermatol 138:57-62.
37. Miller, R. A. 1982. The Koebner phenomenon. Int J Dermatol 21:192-197.
38. Kocak, M., Bozdogan, O., Erkek, E., Atasoy, P., and Birol, A. 2003. Examination of Bcl-2, Bcl-X and bax protein expression in psoriasis. Int J Dermatol 42:789-793.
39. Wrone-Smith, T., Johnson, T., Nelson, B., Boise, L. H., Thompson, C. B., Nunez, G., and Nickoloff, B. J. 1995. Discordant expression of Bcl-x and Bcl-2 by keratinocytes in vitro and psoriatic keratinocytes in vivo. Am J Pathol 146:1079-1088.
40. Elder, J. T., Tavakkol, A., Klein, S. B., Zeigler, M. E., Wicha, M., and Voorhees, J. J. 1990. Protooncogene expression in normal and psoriatic skin. J Invest Dermatol 94:19-25.
41. Osterland, C. K., Wilkinson, R. D., and St Louis, E. A. 1990. Expression of c-myc protein in skin and synovium in psoriasis and psoriatic arthritis. Clin Exp Rheumatol 8:145-150.
42. Levy, D. E., and Darnell, J. E., Jr. 2002. Stats: transcriptional control and biological impact. Nat Rev Mol Cell Biol 3:651-662.
43. Lowes, M. A., Bowcock, A. M., and Krueger, J. G. 2007. Pathogenesis and therapy of psoriasis. Nature 445:866-873.

### Example 5

### CD109 Regulates the Production of ECM in Scleroderma Skin Fibroblasts

The objective of this study was to determine whether CD109 plays a role in regulating fibrotic process in scleroderma (systemic sclerosis, SSc).

### Patients and Methods

Human subjects: Sixteen female patients with SSc from Rheumatology Department, Jewish General Hospital and 9 controls (7 females, 2 males) from patients undergoing plastic surgery at the Montreal General Hospital were studied. The diagnosis of SSc was established according to the classification criteria of the American College of Rheumatology (13). Punch biopsy samples were obtained from the extensor surfaces of the dorsal forearm or abdomen with written informed consent from the subjects. Biopsies were cut into two parts. One half was placed in 10% buffered formalin for immunofluorescence and the other half was put into DMEM medium for fibroblasts isolation. This study was conducted in agreement with the Declaration of Helsinki and under the protocol approved by the institutional review committee of McGill University. Cell culture: Primary dermal fibroblasts were established from excisional skin biopsy samples. Biopsy samples were put into 0.5% dispase (Invitrogen Corporation, Carlsad, Calif.) overnight at 4° C. The dermis was isolated from the epidermis and incubated with 0.1% collagenase type I (Invitrogen) overnight to release fibroblasts which were then cultured in DMEM with 10% FBS (fetal bovine serum) at 37° C. in 5% CO₂. For these experiments, control and SSc fibroblasts were grown simultaneously and studied between passages 3 and 6. When the fibroblasts reached confluence in 6-well plates, fresh medium with indicated concentrations of TGF-β1 (Genzyme Corporation, Framingham, Mass.) or recombinant CD109 (R&D Systems Inc.) with or without 100 pM of TGF-β1 was added after being serum-starved for 24 h. Cell lysates were collected and stored in a −80° C. freezer.

Immunofluorescence: Normal and SSc skin tissue specimens were fixed in formalin, embedded in paraffin and cut into 4-μm serial sections using a microtome. After deparaffinization and rehydration, antigen retrieval was performed by heating in sodium citrate buffer (10 mmol/L, pH 8.5) at 95° C. for 20 min. Normal and SScl fibroblasts cultured on 12 mm round coverslips were fixed with 4% paraformaldehyde and heated in sodium citrate buffer as above. Then, fibroblasts were permeabilized at room temperature for 15 min with PBS containing 0.1% Triton X-100™. Blocking was performed with 10% normal rabbit serum for 1 h at room temperature. Sections were then incubated with 1:2000 dilution of mouse anti-human CD109 antibody (R&D Systems Inc.) overnight at 4° C., followed by fluorescein isothiocyanate (FITC)-conjugated rabbit anti-mouse secondary antibody (AF488™, Invitrogen) for 2 h at room temperature in the dark. Immunofluorescent images were obtained using an Olympus™ microscope. Reverse transcription-polymerase chain reaction (RT-PCR): Total RNA was isolated from fibroblasts with RNeasy Plus Mini Kit™ (Qiagen, Valencia, Calif.) according to the manufacturer's instructions and stored at −80° C. until use. The cDNA was synthesized by using 2 μg of total RNA, 200 U MMLV reverse transcriptase (Invitrogen) and 0.5 μg of oligo(dT)₁₈ as primer in a total reaction volume of 20 μl. Each experiment included samples containing no reverse transcriptase (negative controls) to exclude amplification from contaminating genomic DNA. A primer pair (forward primer: 5′-GCCTTTGATTTAGATGTTGCTGTA-3′ (SEQ ID NO: 7); reverse primer: 5′-TATTCCACTTTCTTCACTGTCTCG-3′ (SEQ ID NO: 8), product length: 188 bp) was designed to amplify CD109. PCR amplification was performed in a volume of 25 μl, which includes 0.5 μl of cDNA mixture, 5 pmol of each primer, 200 mM each dNTP, 2 mM MgCl₂, 10 mM Tris-HCl, pH 8.3, 50 mM KCl, and 2 U of Taq DNA polymerase. After a denaturation at 96° C. for 5 min, the reaction was performed for 25 cycles at 96° C. for 30 s, 59° C. for 30 s, and 72° C. for 30 s, and a final extension for 10 min at 72° C. The PCR products were separated in 1.5% agarose gel, stained with ethidium bromide, and photographed on a UV transilluminator. Results were expressed for each sample as band intensity relative to that of GAPDH (5′-GGGGAGCCAAAAGGGTCATCATCT-3′ (SEQ ID NO: 9)/5′-TTGGCCAGGGGTGCTAAG-3′ (SEQ ID NO: 10), product length: 145 bp).

siRNATransfection Assay: Transient tranfection was used to examine the effect of siRNA mediated CD109 knockdown on ECM production in fibroblasts. Primary normal and SSc fibroblasts were transfected with CD109-specific (Invitrogen; Stealth™ siRNA) or control siRNA using the TranslT-LT1™ transfection reagent (Mirus Bio). A volume of 7.5 μl of TranslT-LT1™ reagent was mixed with 250 μl serum-free DMEM in sterilized tubes. Then, CD109 and control siRNA were added separately to a final concentration of 10 nM and incubated at room temperature for 30 min. The resulting transfection complexes were then incubated with newly trypsinized fibroblasts (1×10⁶ cells) at room temperature for 30 min prior to plating the cells into a well of a 6-well plate that contained 2 ml of fresh DMEM with 10% FBS in each well. After 48 h, cells were cultured in the presence or absence of TGF-β1 at 100 pM for 30 min prior to harvest for Western blot.

Western blot: The fibroblasts monolayers were lysed by modified RIPA buffer (50 mM Tris-HCl, 1% NP-40, 0.25% Na-deoxycholate, 150 mM NaCl, 1 mM EDTA, 1 mM PMSF, 1 mM Na₃VO₄, 1 mM NaF and 1× Roche complete Protease Inhibitor Cocktail). Protein concentrations were determined by Bradford assay (Bio-Rad, Hercules, Calif.). Whole cell lysates (15 μg/lane) were separated by SDS-PAGE under reducing conditions and the resolved proteins were transferred into nitrocellulose membranes (Millipore, Bedford, Mass.). Following blocking with 5% nonfat dry milk in Tris buffered saline-Tween™ at room temperature for 1 h, membranes were incubated overnight with antibodies against CD109 (R&D), pSmad1/5 (Cell Signaling Technology), pSmad2 (Cell Signaling Technology), pSmad3 (Cell Signaling Technology), Smad2 (Cell Signaling Technology), collagen type I (Abcam), fibronectin (BD Biosciences) and CTGF (Santa Cruz) followed by incubation with horseradish peroxidase (HRP)-conjugated secondary antibodies. After washing, immunoblots were developed with enhanced-chemiluminescence (ECL) reagents (GE Healthcare, UK) according to the manufacturer's protocol. β actin (Santa Cruz) was measured as a loading control.

### Results

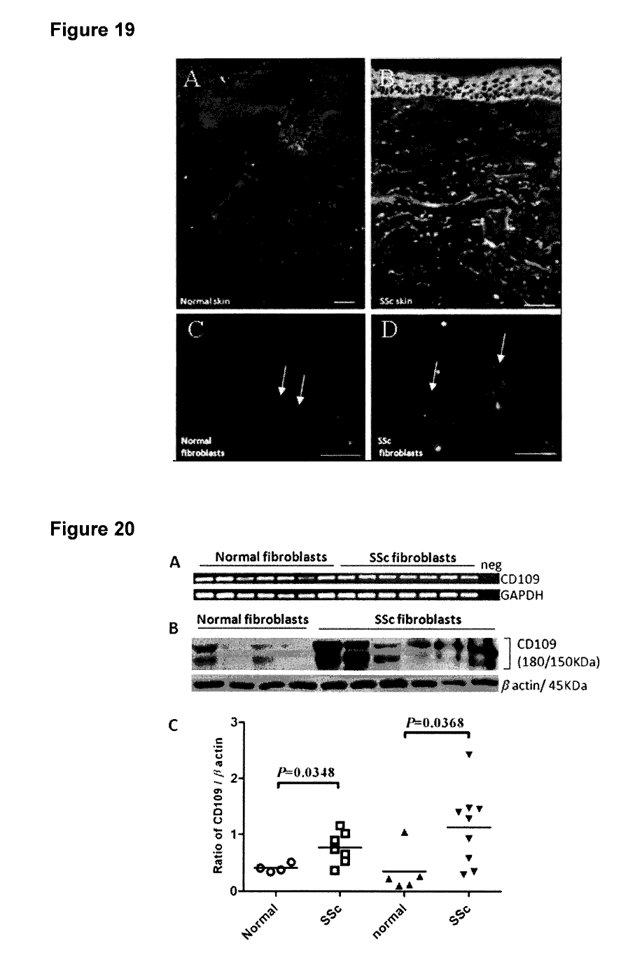

Immunolocalization of CD109 in normal and SSc skin and cultured fibroblasts: In normal and SSc skin, CD109 is localized both in epidermis and dermis (FIG. 19). In the epidermis, CD109 is expressed in the epidermal keratinocytes in all layers except for stratum corneum and mainly localized to the cellular membrane (FIG. 19). In the normal dermis, CD109 is scattered in the whole dermis. However, in SSc dermis, the signal of CD109 is more intense in the middle and deep dermis than in the upper dermis (FIG. 19). To further define the expression of CD109 in dermal fibroblasts, immunofluorescence with anti-CD109 antibody was performed on fibroblasts cultured on coverslips in vitro. CD109 is localized mainly in the cellular membrane and partly in the cytoplasm of normal and SSc fibroblasts (FIG. 19).

CD109 is upregulated at the protein levels but not at the mRNA level in SSc fibroblasts: Total RNA was extracted from fibroblasts obtained from normal and SSc skin. FIG. 20A shows that there is no observable difference of RT-PCR amplified CD109 mRNA between normal and SSc fibroblasts. In contrast, protein levels of CD109 in SSc fibroblasts determined by Western blot are elevated as compared with normal fibroblasts (FIG. 20B). This difference is statistically significant as determined by comparison of the densitometric values of normal and SSc fibroblasts (P<0.05) (FIG. 20C). These results indicate that expression of CD109 may be altered at post-transcriptional level, not at the transcriptional level, in SSc dermal fibroblasts.

CD109 protein is not altered by TGF-β1 in normal and SSc fibroblasts: There are three TGF-β isoforms, TGF-β1, TGF-β2 and TGF-β3 (15). Tissue fibrosis is primarily attributed to the TGF-β1 isoform (16-17). Whether CD109 expression could be regulated by TGF-β1 was evaluated. FIG. 21 shows that TGF-β1 does not alter CD109 protein levels in normal or SSc fibroblasts, although it enhances the production of fibronectin in a dose dependent manner as expected.

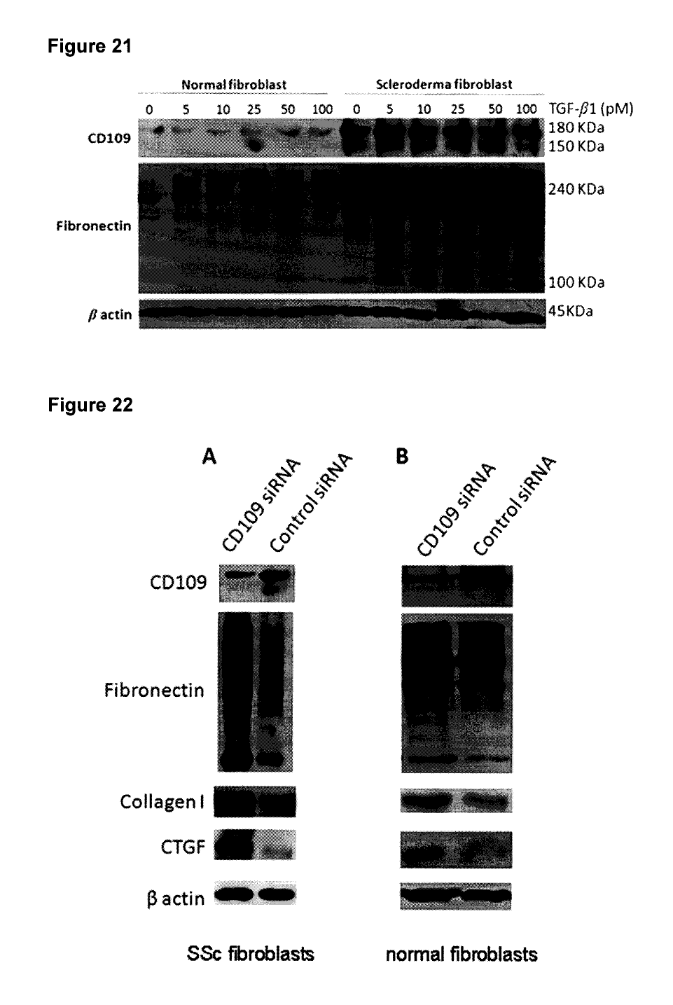

CD109 decreases collagen type I, fibronectin and CTGF expression and Smad2/3 phosphorylation: The potential role of CD109 in the process of SSc dermal fibrosis was examined by a loss of function approach using CD109-specific siRNA. As compared with the control siRNA, the CD109 siRNA decreases the synthesis of CD109 efficiently in normal and SSc fibroblasts (FIG. 22). Moreover, after CD109 levels are decreased, the production of fibronectin, collagen type I and CTGF is increased in both SSc and normal fibroblasts (FIG. 22). That is, CD109 inhibits production of collagen type I, fibronectin and CTGF in both normal and SSc fibroblasts.

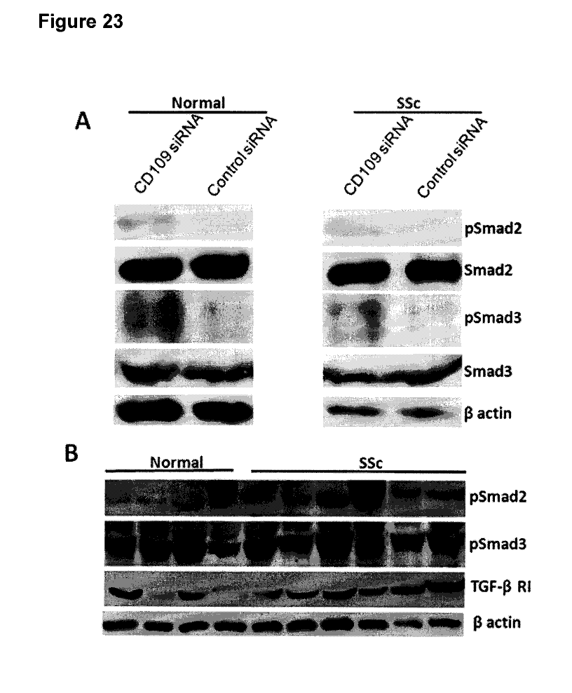

It is well known that ECM production is mainly associated with activation of Smad2 and Smad3 (1, 5-6, 18). Therefore, next, the effect of CD109 siRNA on TGF-β1-induced phosphorylation of Smad2 and Smad3 was examined. The results show that CD109 siRNA increases TGF-β1-induced phosphorylation of Smad2 and Smad3 as compared to control siRNA in both SSc and normal fibroblasts (FIG. 23A).

Smad2 but not Smad3 phosphorylation is increased SSc dermal fibroblasts: The levels of pSmad2 and pSmad3 were further examined in normal and SScl fibroblasts in vitro. Results show that the levels of Smad2 phosphorylation is higher in SSc fibroblasts as compared with normal fibroblasts (FIG. 23B). However, no obvious difference is observed in phospho-Smad3 levels in SSc fibroblasts and normal fibroblasts in vitro. That is, phosphorylation levels of Smad3 detected in these SSc subjects in the present study is not elevated which is in contrast to a previous study reporting elevated pSmad3 levels in SSc fibroblasts (18). This discrepancy may be due to differences in the stage of the disease or in the areas of the biopsy from which the fibroblasts were taken.

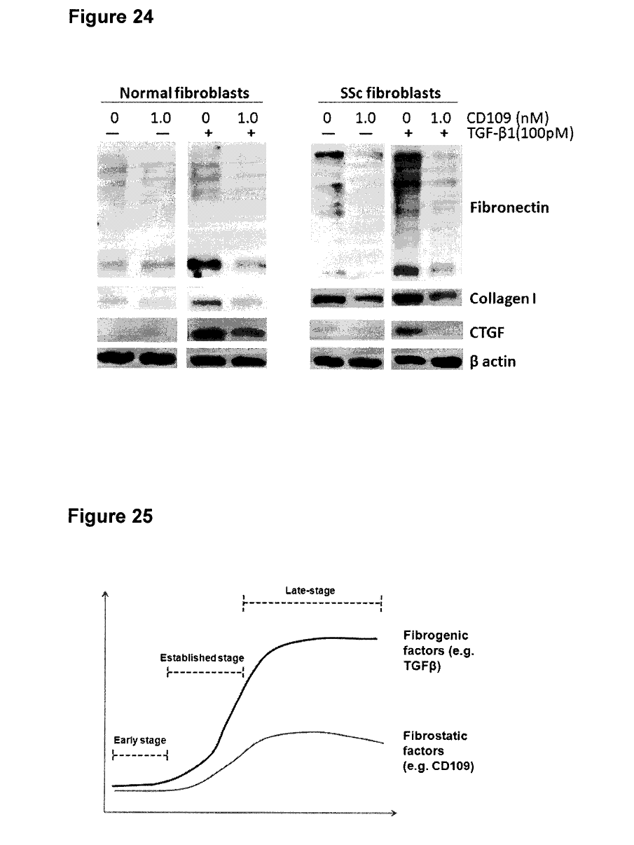

Recombinant CD109 protein decreases production of fibronectin, collagen and CTGF: To further verify the results obtained by siRNA transfection showing that CD109 inhibits production of ECM in normal and SSc fibroblasts, an exogenous recombinant CD109 was used in vitro. Recombinant CD109 protein at 1.0 nM with or without 100 pM of TGF-β1 was added to confluent serum-starved fibroblasts for 24 h in 6-well plates. FIG. 24 shows that recombinant CD109 protein decreases basal (absence of TGF-β1) production of fibronectin, collagen type I and CTGF in SSc fibroblasts and inhibits TGF-β1-induced production of fibronectin, collagen type I and especially, CTGF in both SSc and normal fibroblasts (FIG. 24).

Furthermore, the results shown in FIGS. 19 A-D indicate that scleroderma (SSc) skin displays markedly higher levels of CD109 protein as compared to normal skin in vivo (B versus A) and that SSc skin fibroblasts exhibit elevated CD109 protein levels as compared to normal skin fibroblasts in vitro (D versus C), as detected by immunofluorescence. Furthermore, FIGS. 20B and 20C demonstrate that CD109 protein levels are significantly increased in SSc skin fibroblasts as compared to normal skin fibroblasts as detected by Western Blot analysis. These data showing that CD109 protein expression is markedly higher in SSc skin as compared to normal skin, indicate that high CD109 expression correlate with a SSc phenotype. Thus, CD109 can be of value as a biomarker for SSc.

### Discussion

This study shows that CD109 is expressed in SSc and normal epidermal keratinocytes and dermal fibroblasts. Furthermore, CD109 is consistently upregulated in dermal fibroblasts cultured from involved areas of skin from SSc patients relative to normal fibroblasts. In established dermal fibrosis, the deposition of dense and closely packed collagen fibers occurs throughout the dermis and is often more prominently in the middle to deep reticular dermis (14). The expression of CD109 appears to vary with the degree of fibrosis in SSc skin with high CD109 expression being associated with the more fibrotic regions.

The possibility that autocrine TGF-β might contribute to elevated CD109 protein levels in SSc was then explored by adding TGF-β1 to cultured normal and SSc fibroblasts and by determining CD109 expression levels. Although TGF-β1 increases the production of fibronectin in a dose-dependent manner, it does not alter the protein level of CD109 both in normal and SSc fibroblasts. It seems that TGF-β1 is not a regulator for CD109.

The functional significance of CD109 in ECM production in SSc and normal fibroblasts was next investigated. To determine if CD109 plays a role in ECM production in SSc, a CD109-specific siRNA was transfected. Intriguingly, after expression of CD109 was blocked, an increase of ECM production was observed. Addition of recombinant CD109 was shown to decrease the basal synthesis of fibronectin, collagen I and CTGF in SSc fibroblasts and to decrease TGF-β1-induced production of these proteins in SSc and normal fibroblasts. These data suggest that CD109 may act in SSc fibroblasts to modulate TGF signaling by acting as a negative regulator of ECM production.

To further investigate the role of CD109 in TGF-β signal transduction in SSc, the effect of CD109 siRNA knockdown on TGF-β1-induced phosphorylation of Smad2 and Smad3 was examined. The results demonstrate that CD109 siRNA increases TGF-β1-induced phosphorylation of Smad2 and Smad3, as compared to control siRNA (FIG. 23). These data suggest that CD109 may act to suppress TGF-β1-induced Smad2/3 signaling to inhibit ECM production in SSc fibroblasts.

However, in SSc fibroblasts, although the protein levels of CD109 is upregulated, the phosphorylation levels of Smad2 and Smad3 is not reduced in the SSc subjects studied. For pSmad2, it is even upregulated. These results are contrary to the fact that CD109 acts as a suppressor of Smad2/3 signaling pathway in SSc fibroblasts. The reasons behind this paradox may be that there exist an autoregulation mechanism for the production of ECM; CD109 is increased in the context of SSc representing an adaptive mechanism to inhibit production of ECM. However, this inhibition cannot compensate for those profibrogenic factors effect (FIG. 25). So, the balance in maintaining synthesis of ECM is disrupted in SSc and shifts toward profibrogenic process and ECM production.

In conclusion, this study demonstrates that CD109 is upregulated in SSc skin sections and cultured fibroblasts. CD109 is an important regulator of ECM production in SSc fibroblasts with blocking CD109 expression leading to an increase and addition of recombinant CD109 protein resulting in a decrease in ECM production. CD109 may exert these effects by regulating Smad2/3 activation since blocking CD109 expression leads to an increase in Smad2/3 phosphorylation. Thus, the upregulation of CD109 in SSc may represent an adaptive response to aberrant activation of TGF-β signaling pathways in SSc. Findings that CD109 is able to decrease excessive ECM production in SSc fibroblasts supports a therapeutic value of this molecule for the treatment of SSc.

### REFERENCES

1. Denton C P, Black C M, Abraham D J. Mechanisms and consequences of fibrosis in systemic sclerosis. Nat Clin Pract Rheumatol. 2006; 2(3):134-44.
2. Varga J, Abraham D. Systemic sclerosis: a prototypic multisystem fibrotic disorder. J Clin Invest. 2007; 117(3):557-67.
3. Ihn H. Autocrine TGF-beta signaling in the pathogenesis of systemic sclerosis. J Dermatol Sci. 2008; 49(2):103-13.
4. Leask A. Scar wars: is TGFbeta the phantom menace in SSc? Arthritis Res Ther. 2006; 8(4):213.
5. Pannu J, Trojanowska M. Recent advances in fibroblast signaling and biology in SSc. Curr Opin Rheumatol. 2004; 16(6):739-45.
6. Shi Y, Massague J. Mechanisms of TGF-beta signaling from cell membrane to the nucleus. Cell. 2003; 113(6):685-700.
7. Tam B Y, Finnson K W, Philip A. Glycosylphosphatidylinositol-anchored proteins regulate transforming growth factor-beta signaling in human keratinocytes. J Biol Chem. 2003; 278(49):49610-7.
8. Finnson K W, Tam B Y, Liu K, Marcoux A, Lepage P, Roy S, et al. Identification of CD109 as part of the TGF-beta receptor system in human keratinocytes. FASEB J. 2006; 20(9):1525-7.
9. Solomon K R, Sharma P, Chan M, Morrison P T, Finberg R W. CD109 represents a novel branch of the alpha2-macroglobulin/complement gene family. Gene. 2004; 327(2):171-83.
10. Lin M, Sutherland D R, Horsfall W, Totty N, Yeo E, Nayar R, et al. Cell surface antigen CD109 is a novel member of the alpha(2) macroglobulin/C3, C4, C5 family of thioester-containing proteins. Blood. 2002; 99(5):1683-91.
11. Hasegawa M, Hagiwara S, Sato T, Jijiwa M, Murakumo Y, Maeda M, et al. CD109, a new marker for myoepithelial cells of mammary, salivary, and lacrimal glands and prostate basal cells. Pathol Int. 2007; 57(5):245-50.
12. Hagiwara S, Murakumo Y, Sato T, Shigetomi T, Mitsudo K, Tohnai I, et al. Up-regulation of CD109 expression is associated with carcinogenesis of the squamous epithelium of the oral cavity. Cancer Sci. 2008; 99(10):1916-23.
13. Lonzetti L S, Joyal F, Raynauld J P, Roussin A, Goulet J R, Rich E, et al. Updating the American College of Rheumatology preliminary classification criteria for systemic sclerosis: addition of severe nailfold capillaroscopy abnormalities markedly increases the sensitivity for limited SSc. Arthritis Rheum. 2001; 44(3):735-6.
14. Gabrielli A, Avvedimento E V, Krieg T. SSc. N Engl J Med. 2009; 360(19):1989-2003.
15. Gorelik L, Flavell R A. Transforming growth factor-beta in T-cell biology. Nat Rev Immunol. 2002; 2(1):46-53.
16. Varga J, Pasche B. Transforming growth factor beta as a therapeutic target in systemic sclerosis. Nat Rev Rheumatol. 2009; 5(4):200-6.
17. Wynn T A. Cellular and molecular mechanisms of fibrosis. J Pathol. 2008; 214(2):199-210.
18. Mori Y, Chen S J, Varga J. Expression and regulation of intracellular SMAD signaling in SSc skin fibroblasts. Arthritis Rheum. 2003; 48(7):1964-78.
19. Varga J A, Trojanowska M. Fibrosis in systemic sclerosis. Rheum Dis Clin North Am. 2008; 34(1):115-43; vii.
20. Denton C P, Abraham D J. Transforming growth factor-beta and connective tissue growth factor: key cytokines in SSc pathogenesis. Curr Opin Rheumatol. 2001; 13(6):505-11.
21. Ihn H. Pathogenesis of fibrosis: role of TGF-beta and CTGF. Curr Opin Rheumatol. 2002; 14(6):681-5.
22. Jinnin M, Ihn H, Yamane K, Tamaki K. Interleukin-13 stimulates the transcription of the human alpha2(I) collagen gene in human dermal fibroblasts. J Biol Chem. 2004; 279(40):41783-91.
23. Pannu J, Gardner H, Shearstone J R, Smith E, Trojanowska M. Increased levels of transforming growth factor beta receptor type I and up-regulation of matrix gene program: A model of SSc. Arthritis Rheum. 2006; 54(9):3011-21.
24. Pannu J, Gore-Hyer E, Yamanaka M, Smith E A, Rubinchik S, Dong J Y, et al. An increased transforming growth factor beta receptor type I:type II ratio contributes to elevated collagen protein synthesis that is resistant to inhibition via a kinase-deficient transforming growth factor beta receptor type II in SSc. Arthritis Rheum. 2004; 50(5):1566-77.
25. Yamane K, Ihn H, Kubo M, Tamaki K. Increased transcriptional activities of transforming growth factor beta receptors in SSc fibroblasts. Arthritis Rheum. 2002; 46(9):2421-8.
26. Dharmapatni A A, Smith M D, Ahern M J, Simpson A, Li C, Kumar S, et al. The TGF beta receptor endoglin in systemic sclerosis. Asian Pac J Allergy Immunol. 2001; 19(4):275-82.
27. Leask A, Abraham D J, Finlay D R, Holmes A, Pennington D, Shi-Wen X, et al. Dysregulation of transforming growth factor beta signaling in SSc: overexpression of endoglin in cutaneous SSc fibroblasts. Arthritis Rheum. 2002; 46(7):1857-65.

### Example 6

### Recombinant CD109 Protein and a CD109-Based Peptide (Peptide A) Decrease TGF-β-Induced Production of Extracellular Matrix (ECM) Proteins in Skin Cells

In this Example, the efficacy of recombinant CD109 protein and a peptide based on the putative TGF-β binding region of CD109 (Peptide A, corresponding to amino acid sequence 606-766 of CD109) to bind TGF-β1 and inhibit TGF-β1-induced ECM production in skin cells was explored.

### Experimental

Skin biopsies were obtained from scleroderma (systemic sclerosis, SSc) patients or normal control subjects and skin fibroblasts were isolated and cultured as described previously (Finnson et al., 2006). Primary SSc and normal fibroblast were treated for 18 hours without or with 100 pM TGF-β1 in the presence of 0-1.0 nM of recombinant CD109 protein or 0-5 nM Peptide A as a GST-fusion protein or GST control. Cell lysates were prepared and analyzed by Western blot to detect collagen type I, fibronectin, PAI-1 or CTGF proteins. Membranes were reprobed with anti-β-actin antibody as a loading control.

### Results

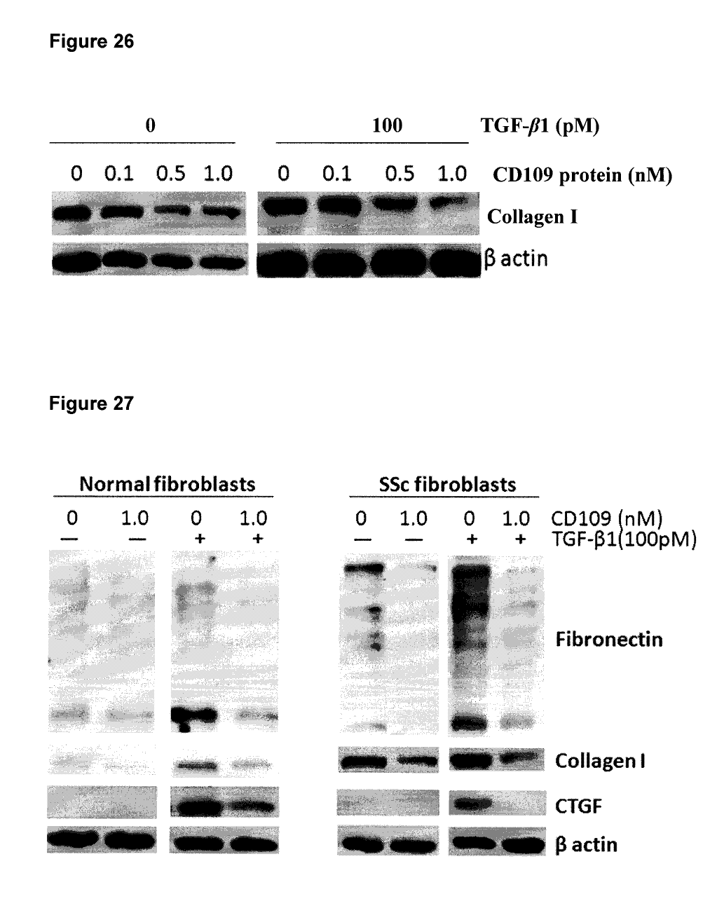

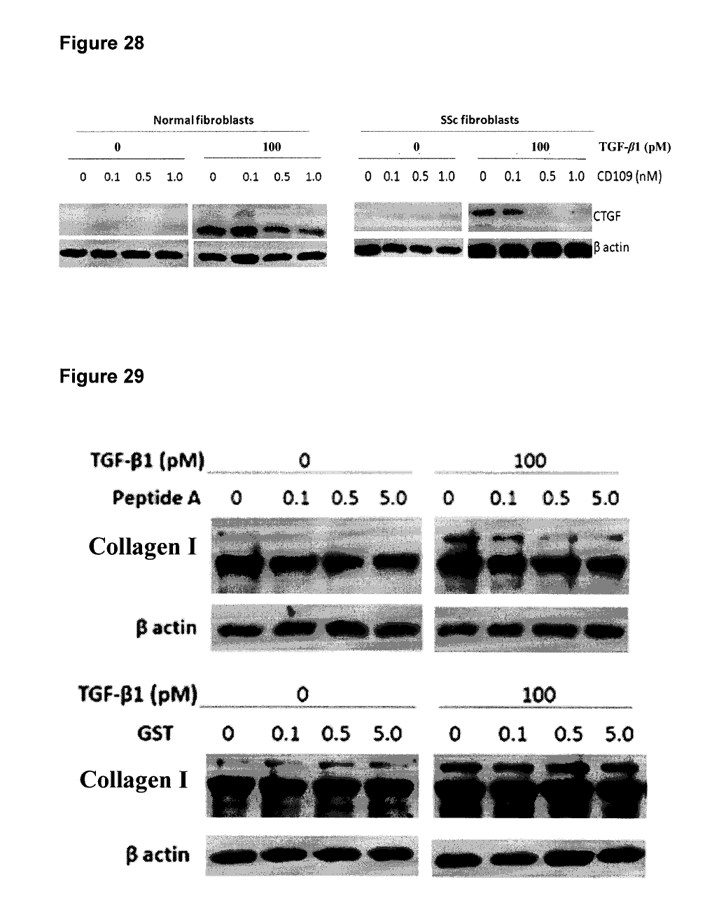

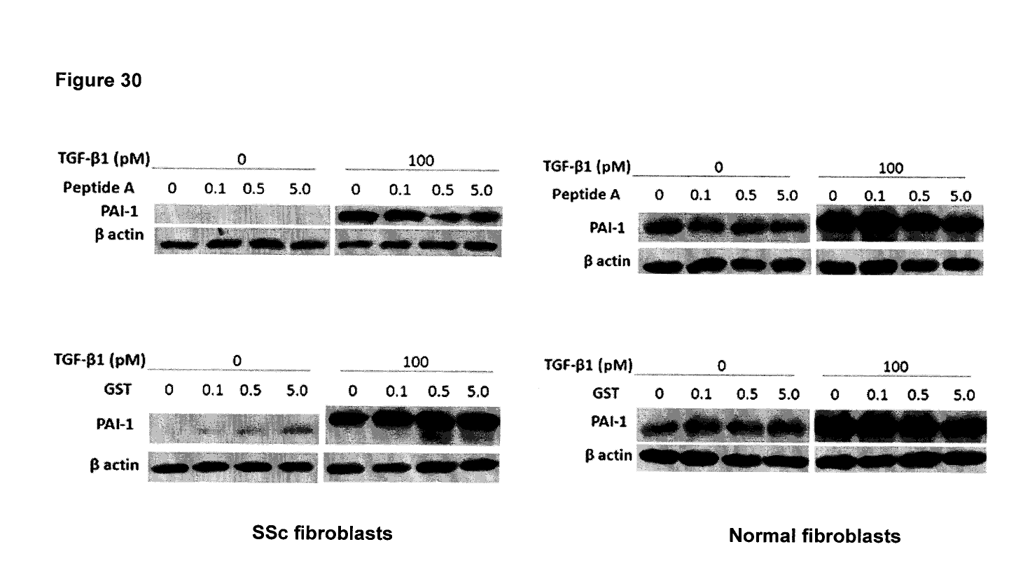

The effect or recombinant CD109 protein on ECM production was first examined in SSc and normal fibroblasts. The results demonstrate that the recombinant CD109 protein inhibits basal and TGF-β1-induced production of collagen type I, fibronectin and CTGF proteins in SSc and normal fibroblasts (FIGS. 26 to 28). Reprobing the membranes with anti-β-actin antibodies reveals that equivalent amounts of proteins were loaded in each lane (FIGS. 26 to 28). Next, the ability of Peptide A to inhibit ECM production in these cells was examined. The results indicate that the CD109 based GST-fusion peptide, Peptide A, but not GST control protein, inhibits basal and TGF-β1-induced collagen type I in SSc fibroblasts (FIG. 29) and basal and TGF-β1-induced PAI-1 protein production in normal fibroblasts (FIG. 30, right panel). Peptide A-GST also inhibits TGF-β1-induced PAI-1 protein expression in SSc fibroblasts (FIG. 30; left panel).

### CONCLUSIONS

These results indicate that soluble CD109 protein and a peptide corresponding to the putative TGF-β1 binding region of CD109 (Peptide A) inhibit TGF-β1-induced production of collagen type I, fibronectin, PAI-1 and CTGF proteins in SSc or normal skin fibroblasts. These findings provide a basis for a novel therapeutic approach in which recombinant CD109 protein or a CD109-based peptide (peptide A) may be used to modulate TGF-β action locally to therapeutically treat atypical scarring, and may also have relevance to diseases such as scleroderma and psoriasis where dysregulation of TGF-β action is implicated.

Headings are included herein for reference and to aid in locating certain sections. These headings are not intended to limit the scope of the concepts described therein under, and these concepts may be applicable in other sections throughout the entire specification. Thus, the present invention is not intended to be limited to the embodiments shown herein but is to be accorded the widest scope consistent with the principles and novel features disclosed herein.

As used herein and in the appended claims, the singular forms “a”, “an”, and “the” include plural referents unless the context clearly indicates otherwise. Thus, for example, reference to “a CD109 polypeptide” includes one or more of such polypeptides, and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. A method for the in vivo treatment of skin cells of a mammalian subject in need thereof, the method comprising administering to said subject a therapeutically effective amount of a CD109 polypeptide.

2. The method of claim 1, wherein said CD109 polypeptide comprises an amino acid sequence selected from the group consisting of SEQ ID NO:2, SEQ ID NO:4, SEQ ID NO:6; and therapeutically active fragments thereof.

3. The method of claim 1, wherein said treatment of skin cells comprises at least one of reducing skin fibrosis, reducing skin scarring and promoting wound healing.

4. The method of claim 1, wherein said subject in need thereof is afflicted with a skin disorder.

5. The method of claim 4, wherein said skin disorder is selected from the group consisting of scarring, hypertrophic scarring, keloid scarring, fibrotic disorder, delayed wound healing, psoriasis and scleroderma.

6. The method of claim 5, wherein said scarring derives from a burn, a trauma, a surgical injury or a chronic condition.

7. The method of claim 1, wherein said mammalian subject is a human.

8. The method of claim 1, wherein said administering comprises contacting said skin cells with a therapeutically effective amount of a CD109 polypeptide.

9.-12. (canceled)

13. The method of claim 3, wherein promoting healing of said wound comprises at least one of the following:
improving collagen organization and/or reducing wound cellularity in said wound;
promoting epidermal thickening and/or inhibiting fibroplasia in said wound;
promoting keratinocyte proliferation and/or inhibiting keratinocyte migration in said wound;
reducing and/or inhibiting dermal thickening in said wound;
reducing granulation and/or promoting resolution of granulation in said wound;
reducing and/or inhibiting recruitment of inflammatory cells to said wound;
decreasing macrophages and/or neutrophils presence in said wound; and
decreasing dermal cellularity and/or inhibiting granulation in said wound.

14. (canceled)

15. The method of claim 6, wherein said chronic condition is selected from the group consisting of diabetic foot ulcers, venous leg ulcers and pressure ulcers.

16. (canceled)

17. The method of claim 1, wherein said in vivo treatment comprises increasing proliferation and/or survival of keratinocytes.

18.-23. (canceled)

24. A composition for application to the skin of a mammalian subject in need thereof, the composition comprising a therapeutically effective amount of a CD109 polypeptide for the treatment of skin cells, and a pharmaceutically acceptable vehicle.

25. A cosmetic composition for application onto the skin of a human subject, the composition comprising a cosmetically acceptable vehicle and a CD109 polypeptide capable of reducing skin fibrosis, reducing skin scarring and/or promoting wound healing.

26. An isolated or purified polypeptide consisting of the amino acid sequence of SEQ ID NO:6.

27. (canceled)

28. (canceled)

29. A method for the diagnosis or monitoring of a skin disorder in a human subject, comprising assessing expression of a CD109 polypeptide in a skin sample from said subject.

30. The method of claim 29, wherein CD109 polypeptide levels are lower in a lesional skin sample from a subject suffering from psoriasis as compared to a normal skin sample from a healthy subject.

31. The method of claim 29, wherein CD109 polypeptide levels are higher in a skin sample from a subject suffering from scleroderma as compared to a skin sample from a normal healthy subject.

32. A non-human transgenic mammal overexpressing a CD109 polypeptide in its epidermis.

33. The non-human transgenic mammal of claim 32, wherein said mammal is a mouse.


SEQUENCE LISTING (10 sequences)

SEQ ID NO: 1 - DNA, 4338 bp, Homo sapiens
  CDS (1)..(4338)
atgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttacaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaaatgaaataaatgccacaggccaccagcagacccttctggttcccagtgaggatggggcaactgttctttttcccatcaggccaacacatctgggagaaattcctatcacagtcacagctctttcacccactgcttctgatgctgtcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctgtaaagtttctgattgacacacacaaccgcttactccttcagacagcagagcttgctgtggtacagccaatggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtga

SEQ ID NO: 2 - protein, 1445 aa, Homo sapiens
MQGPPLLTAAHLLCVCTAALAVAPGPRFLVTAPGIIRPGGNVTIGVELLEHCPSQVTVKAELLKTASNLTVSVLEAEGVFEKGSFKTLTLPSLPLNSADEIYELRVTGRTQDEILFSNSTRLSFETKRISVFIQTDKALYKPKQEVKFRIVTLFSDFKPYKTSLNILIKDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGYRIYQEFEVTVPDSITSWVATGFVISEDLGLGLTTTPVELQAFQPFFIFLNLPYSVIRGEEFALEITIFNYLKDATEVKVIIEKSDKFDILMTSNEINATGHQQTLLVPSEDGATVLFPIRPTHLGEIPITVTALSPTASDAVTQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPVKFLIDTHNRLLLQTAELAVVQPMAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 3 - DNA, 4287 bp, Homo sapiens
  CDS (1)..(4287)
atgcagggcccaccgctcctgaccgccgcccacctcctctgcgtgtgcaccgccgcgctggccgtggctcccgggcctcggtttctggtgacagccccagggatcatcaggcccggaggaaatgtgactattggggtggagcttctggaacactgcccttcacaggtgactgtgaaggcggagctgctcaagacagcatcaaacctcactgtctctgtcctggaagcagaaggagtctttgaaaaaggctcttttaagacacttactcttccatcactacctctgaacagtgcagatgagatttatgagctacgtgtaaccggacgtacccaggatgagattttattctctaatagtacccgcttatcatttgagaccaagagaatatctgtcttcattcaaacagacaaggccttatacaagccaaagcaagaagtgaagtttcgcattgttacactcttctcagattttaagccttacaaaacctctttaaacattctcattaaggaccccaaatcaaatttgatccaacagtggttgtcacaacaaagtgatcttggagtcatttccaaaacttttcagctatcttcccatccaatacttggtgactggtctattcaagttcaagtgaatgaccagacatattatcaatcatttcaggtttcagaatatgtattaccaaaatttgaagtgactttgcagacaccattatattgttctatgaattctaagcatttaaatggtaccatcacggcaaagtatacatatgggaagccagtgaaaggagacgtaacgcttacatttttacctttatccttttggggaaagaagaaaaatattacaaaaacatttaagataaatggatctgcaaacttctcttttaatgatgaagagatgaaaaatgtaatggattcttcaaatggactttctgaatacctggatctatcttcccctggaccagtagaaattttaaccacagtgacagaatcagttacaggtatttcaagaaatgtaagcactaatgtgttcttcaagcaacatgattacatcattgagttttttgattatactactgtcttgaagccatctctcaacttcacagccactgtgaaggtaactcgtgctgatggcaaccaactgactcttgaagaaagaagaaataatgtagtcataacagtgacacagagaaactatactgagtactggagcggatctaacagtggaaatcagaaaatggaagctgttcagaaaataaattatactgtcccccaaagtggaacttttaagattgaattcccaatcctggaggattccagtgagctacagttgaaggcctatttccttggtagtaaaagtagcatggcagttcatagtctgtttaagtctcctagtaagacatacatccaactaaaaacaagagatgaaaatataaaggtgggatcgccttttgagttggtggttagtggcaacaaacgattgaaggagttaagctatatggtagtatccaggggacagttggtggctgtaggaaaacaaaattcaacaatgttctctttaacaccagaaaattcttggactccaaaagcctgtgtaattgtgtattatattgaagatgatggggaaattataagtgatgttctaaaaattcctgttcagcttgtttttaaaaataagataaagctatattggagtaaagtgaaagctgaaccatctgagaaagtctctcttaggatctctgtgacacagcctgactccatagttgggattgtagctgttgacaaaagtgtgaatctgatgaatgcctctaatgatattacaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttggaaataactatattcaattatttgaaagatgccactgaggttaaggtaatcattgagaaaagtgacaaatttgatattctaatgacttcaagtgaaataaatgccacaggccaccagcagacccttctggttcccagtgaggatggggcaactgttctttttcccatcaggccaacacatctgggagaaattcctatcacagtcacagctctttcacccactgcttctgatgctatcacccagatgattttagtaaaggctgaaggaatagaaaaatcatattcacaatccatcttattagacttgactgacaataggctacagagtaccctgaaaactttgagtttctcatttcctcctaatacagtgactggcagtgaaagagttcagatcactgcaattggagatgttcttggtccttccatcaatggcttagcctcattgattcggatgccttatggctgtggtgaacagaacatgataaattttgctccaaatatttacattttggattatctgactaaaaagaaacaactgacagataatttgaaagaaaaagctctttcatttatgaggcaaggttaccagagagaacttctctatcagagggaagatggctctttcagtgcttttgggaattatgacccttctgggagcacttggttgtcagcttttgttttaagatgtttccttgaagccgatccttacatagatattgatcagaatgtgttacacagaacatacacttggcttaaaggacatcagaaatccaacggtgaattttgggatccaggaagagtgattcatagtgagcttcaaggtggcaataaaagtccagtaacacttacagcctatattgtaacttctctcctgggatatagaaagtatcagcctaacattgatgtgcaagagtctatccattttttggagtctgaattcagtagaggaatttcagacaattatactctagcccttataacttatgcattgtcatcagtggggagtcctaaagcgaaggaagctttgaatatgctgacttggagagcagaacaagaaggtggcatgcaattctgggtgtcatcagagtccaaactttctgactcctggcagccacgctccctggatattgaagttgcagcctatgcactgctctcacacttcttacaatttcagacttctgagggaatcccaattatgaggtggctaagcaggcaaagaaatagcttgggtggttttgcatctactcaggataccactgtggctttaaaggctctgtctgaatttgcagccctaatgaatacagaaaggacaaatatccaagtgaccgtgacggggcctagctcaccaagtcctcttgctgtggtacagccaacggcagttaatatttccgcaaatggttttggatttgctatttgtcagctcaatgttgtatataatgtgaaggcttctgggtcttctagaagacgaagatctatccaaaatcaagaagcctttgatttagatgttgctgtaaaagaaaataaagatgatctcaatcatgtggatttgaatgtgtgtacaagcttttcgggcccgggtaggagtggcatggctcttatggaagttaacctattaagtggctttatggtgccttcagaagcaatttctctgagcgagacagtgaagaaagtggaatatgatcatggaaaactcaacctctatttagattctgtaaatgaaacccagttttgtgttaatattcctgctgtgagaaactttaaagtttcaaatacccaagatgcttcagtgtccatagtggattactatgagccaaggagacaggcggtgagaagttacaactctgaagtgaagctgtcctcctgtgacctttgcagtgatgtccagggctgccgtccttgtgaggatggagcttcaggctcccatcatcactcttcagtcatttttattttctgtttcaagcttctgtactttatggaactttggctgtga

SEQ ID NO: 4 - protein, 1428 aa, Homo sapiens [sequence omitted]

SEQ ID NO: 5 - DNA, 483 bp, Homo sapiens
  CDS (1)..(483)
acaatggaaaatgtggtccatgagttggaactttataacacaggatattatttaggcatgttcatgaattcttttgcagtctttcaggaatgtggactctgggtattgacagatgcaaacctcacgaaggattatattgatggtgtttatgacaatgcagaatatgctgagaggtttatggaggaaaatgaaggacatattgtagatattcatgacttttctttgggtagcagtccacatgtccgaaagcattttccagagacttggatttggctagacaccaacatgggttccaggatttaccaagaatttgaagtaactgtacctgattctatcacttcttgggtggctactggttttgtgatctctgaggacctgggtcttggactaacaactactccagtggagctccaagccttccaaccatttttcatttttttgaatcttccctactctgttatcagaggtgaagaatttgctttg

SEQ ID NO: 6 - protein, 161 aa, Homo sapiens
TMENVVHELELYNTGYYLGMFMNSFAVFQECGLWVLTDANLTKDYIDGVYDNAEYAERFMEENEGHIVDIHDFSLGSSPHVRKHFPETWIWLDTNMGSRIYQEFEVTVPDSITSWVATGFVISEDLGLGLTTTPVELQAFQPFFIFLNLPYSVIRGEEFAL

SEQ ID NO: 7 - DNA, 24 bp, Artificial Sequence
  Sequence is a completely synthesized primer
gcctttgatttagatgttgctgta

SEQ ID NO: 8 - DNA, 24 bp, Artificial Sequence
  Sequence is a completely synthesized primer
tattccactttcttcactgtctcg

SEQ ID NO: 9 - DNA, 24 bp, Artificial Sequence
  Sequence is a completely synthesized primer
ggggagccaaaagggtcatcatct

SEQ ID NO: 10 - DNA, 18 bp, Artificial Sequence
  Sequence is a completely synthesized primer
ttggccaggggtgctaag